(19) United States
(12) Patent Application Publication
(10) Pub. No.: US 2021/0298890 A1
(43) Pub. Date: Sep. 30, 2021

(54) IMPLANTS FOR RECRUITING AND REMOVING CIRCULATING TUMOR CELLS

(71) Applicant: Cirlo GmbH, Wolfratshausen (DE)

(72) Inventors: Sabine BAUER, Wolfratshausen (DE); Thomas ISCHINGER, München (DE)

(73) Assignee: Cirlo GmbH, Wolfratshausen (DE)

(21) Appl. No.: 17/255,900

(22) PCT Filed: Jun. 27, 2019
(86) PCT No.: PCT/EP2019/067269

§ 371 Date: Dec. 23, 2020

(30) Foreign Application Priority Data
Jun. 27, 2018 (EP) 18180045.9

(51) Int. Cl. A61F 2/01; C07K 14/705

(52) U.S. Cl. CPC A61F 2/0105; A61F 2/0108; A61F 2/011; A61F 2250/0098; C07K 14/705; A61F 2002/016; A61F 2250/0067; A61F 2/012

(57) ABSTRACT

The present invention relates to a filter device assembly implant comprising one or more chemical and/or biological agents wherein the implant is capable of recruiting a circulating tumor or circulating metastatic cell or motile parts of tumor cells and hereby removes said cell or motile part thereof from circulation. For this purpose the implant comprises an agent, which is capable of binding to a tumor marker. The present invention further relates to the use of the implant for the treatment of cancer or metastasis, or for preventing cancer or metastasis, as well as corresponding methods of treatment. Also envisaged is a method of manufacturing the filter device assembly implant.

### FIELD OF THE INVENTION

The present invention relates to a filter device assembly implant comprising one or more chemical and/or biological agents wherein the implant is capable of recruiting a circulating tumor or circulating metastatic cell or motile parts of tumor cells and thereby removes said cell or motile part thereof from circulation. For this purpose the implant comprises an agent, which is capable of binding to a tumor marker. The present invention further relates to the use of the implant for the treatment of cancer or metastasis, or for preventing cancer or metastasis, as well as corresponding methods of treatment. Also envisaged is a method of manufacturing the filter device assembly implant.

### BACKGROUND OF THE INVENTION

According to the World Health Organisation, cancer represents the second most important cause of death and morbidity in Europe with more than 3.7 million new cases and 1.9 million deaths each year. On a global scale, cancer accounted for 8.2 million deaths (around 13% of the total) in 2012. Tobacco consumption and excessive alcohol consumption cause about 40% of the total cancer burden. Although more than 40% of cancer deaths can theoretically be prevented, cancer still accounts for 20% of deaths in the European Region. While Europe comprises only one eighth of the total world population it has around one quarter of the global total of cancer cases. The most cancer deaths each year are caused by lung, breast, stomach, liver, colon and breast cancer cause. Cancer thus remains a key public health concern and a tremendous burden on EU societies since it is the second largest cause of death in the European Union. According to the US National cancer institute the number of people living beyond a cancer diagnosis reached nearly 14.5 million in 2014 and is expected to rise to almost 19 million by 2024. More importantly, approximately 39.6 percent of men and women will be diagnosed with cancer at some point during their lifetimes, based on 2010-2012 data.

One defining feature of cancer is the rapid creation of abnormal cells that grow beyond their usual boundaries, and which can then invade adjoining parts of the body and spread to other organs, the latter process is referred to as metastasizing. Metastases are a major cause of death from cancer.

Metastasis to distant organs is an ominous feature of most malignant tumors but the natural history of this process varies in different cancers. The cellular origin, intrinsic properties of the tumor, tissue affinities and circulation patterns determine not only the sites of tumor spread, but also the temporal course and severity of metastasis to vital organs. Tumor progression towards metastasis is often depicted as a multistage process in which malignant cells spread from the tumor of origin to colonize distant organs (Christofori, Nature 441, 444-450, 2006). A salient feature of metastasis is the ability of different tumor types to colonize the same or different organ sites. For example, prostate cancer metastasis is largely confined to bone and metastasis by ocular melanoma is almost exclusively confined to the liver (Edlund et al, J. Cell. Biochem, 91, 686-705, 2004; Triozzi et al., Cancer Treat. Rev., 34, 247-258, 2008). Adenocarcinomas of the breast and lung typically relapse within a similar range of organs, including bone, lung, liver and brain. Breast cancer recurrences are often detected following years or decades of remission, whereas lung cancers establish distant macrometastases within months of diagnosis (Nguyen et al., Nature Reviews Cancer, 9, 274-284, 2009).

In contrast to tumor types that rapidly colonize distant organs with a short disease-free interval on initial diagnosis there are tumors that can efficiently infiltrate distant organs at early stages but are unable to promptly grow as macrometastases. In breast cancer, disseminated tumor cells (DTCs) enter a state of metastatic latency, which is defined as the time between primary tumor diagnosis and clinically detectable metastatic relapse. Malignant cells from breast tumors that disseminate early can reside as single cells or as micrometastatic clusters, as shown in studies of bone marrow samples from patients without overt metastatic disease. These DTCs either lack the ability to colonize or are prevented from displaying colonization by the environment. As a result, DTCs can enter a state of proliferative dormancy by exiting the proliferative cycle for an indefinite period (Nguyen et al., Nature Reviews Cancer, 9, 274-284, 2009). It has further been found that the presence of DTCs in patients whose primary tumors have been removed correlates with metastatic relapse, suggesting that these cells are a source of future recurrence (Braun et al., N. Engl. J. Med., 353, 793-802, 2005). DTCs have, for example, been detected primarily in the bone marrow but also in the peripheral blood and lymph nodes. The lack of specific markers and the difficulty of isolating DTCs from other organs renders unclear, whether these cells widely disseminate or the bone marrow preferentially acts as an initial reservoir of DTCs. It is further uncertain whether metastatic outgrowth preferentially occurs from these earliest latent DTCs or initiates from a later seeding of cancer cells that had already become more aggressive in the context of the expanding primary tumor. It could be shown that overt metastases and most aggressive primary tumors share similar gene expression patterns, implying that at least some metastatic traits are common between metastases and their primary tumor of origin at some stage (Nguyen et al., Nature Reviews Cancer, 9, 274-284, 2009).

The treatment options for metastases mainly focus on the provision of antibody or small molecule medicaments. For example, WO 2017/023994 discloses small molecule compounds, which are capable of binding to urokinase-type plasminogen activator receptor on the surface of metastatic cells and thereby recruit antibodies to the cancerous cell. The targeted cells are subsequently destroyed.

In WO 2005/014006 a combination of bisphonates and Cathepsin K inhibitors is disclosed for the prevention and treatment of bone metastases is disclosed.

In WO 2017/009837 a micro-RNA based treatment approach for solid tumors and metastases is presented, in which miR-96 and miR-182 RNA molecules are administered to patients.

Document US 2012/0208770 discloses a tumor targeting strategy based on the peptide CHP (Carcinoma Homing Peptide) which was able to inhibit metastatic tumor growth in a lung metastasis model.

However, in the currently used metastatic therapy approaches, early forms of metastatic cells, disseminated tumor cells or circulating cells with metastatic potential typically escape treatment attempts due to mal-focused administration schemes and a relatively low concentration and incidence of these cells in the vascular system.

There is thus a need for an alternative therapeutic approach, which allows to trap and eradicate this group of highly dangerous and evasive cells.

### OBJECTS AND SUMMARY OF THE INVENTION

The present invention addresses this need and provides a filter device assembly implant comprising one or more chemical and/or biological agents wherein the implant is capable of recruiting a circulating tumor or circulating metastatic cell or motile parts of tumor cells and thereby removes said cell or motile part thereof from circulation. The present inventors have surprisingly found that the use of an implant which is capable of recruiting circulating tumor cells or circulating metastatic cells or motile parts thereof allows to capture these cells or parts thereof from bodily fluids, in particular from the bloodstream or lymphatic fluids. The thus bound cells or particles are removed from circulation and are therefore no longer capable of depositing at downstream organs or tissues. The cells' presence in the implant further allow for their destruction or disenablement by toxins or pro-apoptotic elements. In addition, the cells can be modified by interaction with molecules present in or on the device leading to the presentation of signs of apoptosis by the cells which results in immune cell attacks. This innovative concept thus facilitates a therapeutic, as well as preventive advancement against circulating tumor cells and in particular circulating metastatic cells, which are otherwise hardly detectable. An additional advantage of the inventive implant is its perioperative and post-operative usability for an efficient capturing and elimination of circulating cancerous cells, which are detached or loosened during tumor resections. By capturing these cells, a new settling of such cells can be effectively prevented, thus significantly increasing the success rate of the surgery. Moreover, the local and selective capture of circulating tumor cells and motile parts thereof in a chosen target vessel advantageously allows the implant to be inserted into the body by a routine minimally invasive catheter procedure, for example after puncture of the target vessel or a vessel leading to the target. Also the replacement of certain components of the implant, in particular the filter membranes, allows for a very efficient use over a prolonged period of time. The present invention's approach thus significantly improves the therapy and prognosis of cancer patients with risk of metastatic disease or manifest metastatic disease.

Vascular stent and filter technology, which resembles the presently claimed approach on a mechanistic level, is well established. A stent usually is a permanent implant into a diseased and obstructed artery or vein in order to improve blood flow and maintain patency of the formerly obstructed vessel. Stents may, for example, be balloon expandable or self-expandable. Balloon expandable stents are typically made of plastically deformable material such as 316 L steel or cobalt-chrome alloys, and are mounted on a deflated balloon, positioned in the target zone and expanded by inflation of the balloon. Self-expandable stents are usually, but not exclusively, made of a nitinol mesh, i.e. an elastically deformable, memory metal, which is held constraint on a catheter by an outer constraining mechanism and is released in the target organ by withdrawal of the constraining tube. The self-expanding stent then takes its predetermined shape via the memory metal effect.

In addition, there are examples of capturing devices such as the Filterwire (Boston Scientific) or Spider FX (Covidien) device (see also FIG. 28), which are essentially filters resembling umbrellas on a wire with uniform pores of typically 80 μm-110 μm in diameter. Their purpose is the capture of macroscopically visible, large size debris during an interventional procedure. At termination of the procedure the filters are withdrawn. Also known in the art is the so called vena cava filter (Cook, Crux Biomedical), which is a crude filter device that is released in the Vena cava in patients with extremely high risk of spontaneous venous thromboembolism. The purpose of this filter is to trap large masses of thrombotic material in order to avoid pulmonary embolism. While it is intended for permanent implantation, it may be retrieved by lasso or other known catheter techniques as long as it is not overgrown by tissue or not perforated into the vessel wall. Recently, a non permanent vena cava embolic filter has been introduced for the prevention of pulmonary embolism in high risk medical situations in critically ill patients, i.e. during trauma surgery and when anticoagulation is contraindicated (Angel catheter, Bio2Medical).

There is, however, no prior art disclosure of a dedicated filter device assembly implant, which is specially designed for non-permanent use and for being capable of recruiting a circulating tumor or circulating metastatic cell or motile parts of tumor cells and thereby removes said cell or motile part thereof from circulation, featuring, for example, inter alia self-expansion and antimigratory mechanical properties, clogging resistance according to the present invention. Thus, while resembling some aspects of traditional stents and conventional embolic filters, the filter device assembly implant of the present invention advantageously transforms some of the physical features of traditional stent and emboli (blood clots) preventing filter technology into pharmaceutical composition-like devices which are capable of fulfilling completely different purposes such as tumor cell scavenging.

Another advantage and a primary goal of the presently envisaged implant is that the device, the filter component of which is aiming at cell and not emboli or debris capture, thus featuring significantly smaller filter structures or pore sizes is designed to maintain blood flow even through the microscopically small filter structures by creating areas with unhindered flow within or adjacent to the filter membranes in order to minimize reduction or stasis of flow or thrombosis. This approach is in clear contrast to filters known in the art, which are typically intended to completely cover the cross-section of a vessel in order to provide full embolic protection

In a preferred embodiment said implant has one or more of the following properties: (i) it is catheter based; (ii) it is freely positionable in a target vessel, preferably in a minimal invasive manner; (iii) it is retrievable, preferably by catheter means and/or in a minimal invasive manner; (iv) it is anchorable in a target vessel; (v) is designed to fit into and be connected to a permanent implant present in a target vessel as a shuttle docking to a receiving site.

In a further preferred embodiment, the filter device assembly implant comprises (i) a reversible expandable device body having a proximal and a distal end and (ii) at least one filter membrane, preferably characterized by the presence of pores.

In a further preferred embodiment, said filter membrane has at least one of following properties: (i) the filter membrane is expandable and attached to and arranged with the device between its proximal and distal end; (ii) the filter membrane is a non permanent, retrievable filter membrane; (iii) the filter membrane is self-expandable.

In a particularly preferred embodiment said pores have a pore diameter which ranges from about 7 μm to about 100 μm, preferably in a differential manner.

In a further preferred embodiment said filter membrane is at least partially coated with said one or more chemical and/or biological agents.

In a further preferred embodiment said implant is provided in a tubular, onion like, pearl-chain-like, or a birds-nest like shape, or in any mixture of these shapes. In a specific embodiment the multitude of pearl or onion like device configurations of the implant may be of different diameters with at least one of the pearl or onion like configurations in contact with the luminal wall.

In a yet another embodiment, the pearl or onion like configurations of the device hold differential membranes as to the percentage of coverage of the luminal cross-sectional area and their pore diameter and pore pattern.

In a further particularly preferred embodiment said tubular shape is provided by a memory shaped spiraling wire, which forms a tubular spiral.

In a further preferred embodiment, the memory shaped spiraling wire is modified into a single spiral-like wire structure, characterized by incomplete wire circles and an interdigiting structure.

In a further preferred embodiment said onion-like or pearl-chain-like shape is provided by an elastic memory shape meshwork.

In a further preferred embodiment said implant is composed of, is partially composed of, or comprises structural support material selected from the group comprising (i) metal, such as stainless steel, gold, titanium, gold-titanium alloy, cobalt-chromium alloy, tantalum, platinum-radium alloy, tantalum alloy, magnesium, nickel-titanium alloy, e.g. nitinol, silver or copper, (ii) plastic or polymeric material, (iii) elastic memory shape meshwork material such as memory shape elastic wires or (iv) a material readable by tomography or other imaging techniques, e.g. X ray and wherein the device body is not biodegradable or composed of biomaterial or biodegradable material.

In a further preferred embodiment at least a portion of the implant can be activated by balloon inflation.

In a further preferred embodiment said implant comprises at least one docking element at the proximal end for retrieval.

In a further preferred embodiment said implant comprises a radiopaque marker. It is particularly preferred that said radiopaque marker is located at the proximal and distal end, or on at least two opposite portions of the outermost structural element of the device body, allowing to judge radial expansion under medical imaging.

In a further preferred embodiment said filter membrane is composed of elastic, foldable polymer material such as polyurethane, or of micromeshes comprising ultrathin wires, metallic or polymeric material.

In a further preferred embodiment at least one cross-sectional area of the tubular device body is at least partially covered by said filter membrane.

In a further preferred embodiment the plane of the filter membrane is arranged perpendicular to the direction of the longitudinal axis of the device body, or wherein the plane of the filter membrane is in an angle which is non perpendicular to the longitudinal axis of the device body.

In a further preferred embodiment said implant comprises at least two membranes each of which incompletely covers the cross-sectional area and which are arranged in tandem position along the longitudinal axis of the device body, preferably opposite to each other within the circumference of the device body or shifted in clockwise orientation in case of more than 2 membranes in tandem position.

In a further preferred embodiment said implant comprises alternating non-completely covering filter membranes, preferably in a pearl-chain, onion type or birds-nest shape.

In a further preferred embodiment said filter membranes have differential pore diameters and/or differential pattern, preferably ranging from about 7 μm to about 100 μm, or wherein two or more filter membranes have differential pore diameters and/or differential pattern, preferably ranging from about 7 μm to about 100 μm.

In a further preferred embodiment the implant additionally comprises a retrievable embolic filter, preferably with a pore diameter of >100 μm.

In a further preferred embodiment said filter membrane is fully or partially coated on its interior side; or on its exterior side; or on both sides with said one or more chemical and/or biological agents; or wherein said coating differs between different filter membranes. In a specific embodiment, the passive or active coatings may differ between filter membranes, in particular in tandem arrangement of filter membranes each membrane may exhibit different active or passive properties.

In a further preferred embodiment said coating is a passive coating with one or more polymeric materials such as ethylene vinyl acetate (EVA), latexes, urethanes, polyurethanes, polysiloxanes, styrene-ethylene/butylene styrene block copolymers (SEBS), polytetrafluoroethylene (PTFE) or linear aliphatic polyesters.

In an additional, preferred embodiment said passive coating adheres to the structural support material via an adhesive layer, preferably of sugar, starch, polyvinyl-alcohol or degradable products of these materials.

In a particularly preferred embodiment said one or more chemical and/or biological agents constitute an extracellular matrix-like structure.

In a further particularly preferred embodiment said chemical and/or biological agents constituting an extracellular matrix-like structure are selected from the group comprising proteoglycans, such as heparan sulfate, chondroitin sulfate and/or keratin sulfate; non-proteoglycan-polysaccharides such as hyaluronic acid; collagen; elastin; fibronectin and laminin, or a mixture thereof; preferably a protein mixture secreted by Engelbreth-Holm-Swarm (EHS) mouse sarcoma cells, Matrigel, BioCoat or GelTrex.

In an additional, preferred embodiment said implant provides an environment for circulating metastatic cells.

In an additional, preferred embodiment said implant comprises a biological agent as an active coating, which is capable of binding to a tumor marker.

In yet another embodiment, said tumor marker is specific for breast tumors, prostate tumors, pancreas tumors, colon tumors, small cell lung tumors, lymphoma, multiple lymphoma, T-cell tumors, Mycosis fungoides, Melanoma, neuroblastoma, sarcoma, fibrosarcoma, Wilms tumor or Squamous cell carcinoma.

In a further preferred embodiment, said tumor marker is CCR4, CCR6, CCR7, IGF, LFA-1, VLA-4, VLA-5, CD44, CD44 v4-v7, CD44 v6-v7, CD44 D3 (v6-v7), CD44-R (v8-v10), CD44 v10, CD-44R1, CXCR3, CXCR4, CXCR5, CXCR6, CXCR7, Surface Fibronectin, PECAM-1 (CD31), CAM 120/180, Integrin alphaᵥ beta₅, P-Selectin, L-Selectin, Integrin alphaᵥ beta₅, Integrin alpha₄ beta₇, Integrin alpha₂ beta₁, Integrin alpha₂ beta₃, Integrin alphaᵥ beta₃, Galectin-3, N-CAM, L-Selectin, LPAM-I (alpha₄ beta₂), CTLA, Integrin alpha₄ beta₁, Integrin alpha_{E} beta₇, CCR10, Axl/Mer, Anxa2-R or Desmoglein I (DG I).

In a further embodiment, said biological agent which is capable of binding to a tumor marker is selected from one or more of the group comprising a tumor-marker specific antibody or a fragment thereof, CD133 or a fragment or domain thereof, VEGFR-1 or a fragment or domain thereof, a homing factor or a fragment or domain thereof; and a tumor-marker specific lectin or a fragment or domain thereof.

In another preferred embodiment, said homing factor is Osteopontin, Hyaluronate, CXCL12, CCL21, Dipeptidyl Dipeptidase IV, PECAM-1, Bone Sialoprotein, Peripheral Node Addressin (CD34), MADCAM-1, VCAM-1, Collagen type I, Fibronectin, Osteonectin, N-CAM, FGF Receptor, GlyCAM-1, ICAM-1, ICAM-2, ICAM-3, E-Selectin, E-Cadherin, HECA-452, CCL27, CXCL9 (Mig), SDF-1, CXCL16, GAS-6, Anxa2, T140 or CXCL10 (IP10).

It is also preferred that the biological agent is linked to the passive coating of the implant or to the structural support material via a spacer element.

In another embodiment, said linkage to the structural support material is binding to a metal ion resin, such as ion-NTA or ion-agarose.

In yet another preferred embodiment, the spacer element is composed or partially composed of a peptide or polypeptide, preferably the Fc part of an antibody or multi-histidine tag; a nucleic acid; a modified nucleic acid; or a polymer such as PEG, PLA, PVA, polyethylene or polypropylene. In further embodiments, said has a length of about 1 to 20 nm.

The present invention also envisages embodiments, wherein said spacer elements are provided in a density of 2 to 500 per μm² on the surface of the implant.

In an additional embodiment, said biological agent comprises, essentially consists of, or consists of a binding domain capable of binding to a tumor marker.

In yet another preferred embodiment, said binding domain is peptide or polypeptide molecule having a length of about 20 to about 250 amino acids, preferably of about 20 to about 120 amino acids. Particularly preferred is a length of 20, 25, 30, 35, 40, 45, 50, 55, 60, 65, 70, 75, 80, 85, 90, 95, 100, 105, 110, 115 or 120 amino acids.

In a preferred embodiment of said use the homogenization takes place during the process of concentrating macromolecules from said solution via filtration across a membrane present in the concentrator device.

It is also envisaged in specific embodiments that the agent additionally comprises one or more functional domains.

In further embodiments said further functional domain is or comprises, partially comprises or consists of an apoptosis inducing factor or a functional domain of an apoptosis inducing factor capable of inducing apoptosis.

In yet another preferred embodiment said apoptosis inducing factor is FasL/CD95L, TNF-alpha, APO3L or APO2L/TRAIL.

It is also envisaged that said domain capable of binding to a tumor marker and said domain capable of inducing apoptosis are provided as fused domains or are linked via a liner element of about 1 to 20 amino acids length.

According to another embodiment said biological agent is covalently or noncovalently connected to said spacer.

In a further embodiment said connection is a linker element, preferably a peptide having a length of 1, 2, 3, 4, 5, 6, 7, 8, 9, 10, 11, 12, 13, 14, 15, 16, 17, 18, 19 or 20 amino acids.

In a specific embodiment said biological agent is provided as linear or circular element or as an element composed of linear and circular parts, preferably as a linear or circular or partially linear/circular peptide or polypeptide.

In another embodiment said circular biological agent has or is part of a structure comprising a loop or a loop and a stem; or of a linear structure which is linked to said spacer element.

In yet another embodiment, said loop structure or linear structure comprises said biological agent at an exposed position allowing for the binding to a tumor marker or a tumor cell.

In a further preferred embodiment said biological agent comprises or is linked to one or more additional elements selected from the group comprising sugar, branched or unbranched multiple sugar structures, alkynes, azides, streptavidin, biotin, amines, carboxylic acids, active esters, epoxides and aziridines.

In another preferred embodiment, said implant further comprises a pharmaceutical agent, preferably selected from the group comprising an antiproliferative agent and an anticoagulant. It is particularly preferred that said antiproliferative agent is paclitaxel, sirolimus, or an analogue thereof. It is also particularly preferred that said anticoagulant is reteplase or heparin.

In a further aspect the present invention relates to a filter device assembly implant as defined herein above for use in treating cancer and/or metastasis.

In another aspect the present invention relates to a filter device assembly implant as defined herein above for use in preventing cancer and/or metastasis.

In a preferred embodiment of the filter device assembly implant for use as defined above said implant is capable of quantitatively capturing circulating tumor or metastatic cells, preferably circulating metastatic cells, in a subject's body.

In another embodiment said implant is capable of preventing downstream organs or tissues to be reached by tumor or metastatic cells, preferably metastatic cells, which are circulating in a subject's body.

In yet another embodiment said cancer is colon cancer, breast cancer, lung cancer, melanoma, esophageal cancer, prostate cancer, pancreatic cancer, ovarian cancer, myeloma, lymphoma such as ALL, CLL, AML.

In a further preferred embodiment said metastasis is derived from a colon tumor, breast tumor, lung tumor, e.g. small cell lung tumors, squamous cell carcinoma melanoma, prostate tumor, pancreas tumor, lymphoma, T-cell tumor such as Mycosis fungoides, neuroblastoma, sarcoma, fibrosarcoma, ovarial tumor or nephroblastom such as Wilms tumor.

In an embodiment of the filter device assembly implant as defined herein above, or of the filter device assembly implant for use as defined herein above, said implant is designed to be implanted into a blood vessel such as an artery, an elastic artery, a distributing artery, an arteriole, a capillary, a venule or a vein.

In is particularly preferred that said implant is implanted in a blood or lymphatic vessel downstream of an existing cancer site in a subject. Also preferred is that said implant is implanted in close proximity to said existing cancer site.

In another preferred embodiment said implant is implanted in a blood vessel upstream of a tissue with a high risk of developing metastasis.

In yet another preferred embodiment said implant is implanted during and/or after the treatment of a subject with a therapeutic agent or during and/or after surgery removing a tumor load.

In a further embodiment the treatment as mentioned above is an anti-cancer therapy.

In a particular embodiment, said implant is implanted into a healthy subject or a subject showing no symptoms of a disease, preferably symptoms of cancer or metastasis. Preferably, said implant is implanted into a subject being at risk of developing cancer and/or metastasis.

In another aspect the present invention relates to a method of treating cancer and/or metastasis, comprising implanting a filter device assembly implant as defined herein above into a subject in need thereof.

In yet another aspect the present invention relates to a method of preventing cancer and/or metastasis, comprising implanting a filter device assembly implant as deco fined herein above into a healthy subject or a subject being at risk of developing cancer and/or metastasis.

In yet another aspect the present invention relates to a method of manufacturing a filter device assembly implant as defined herein above.

In a preferred embodiment said method comprises the step of providing a biological agent as defined herein above by expressing said biological agent as polypeptide in a suitable host cell, optionally further modifying the polypeptide by adding one or more elements as defined above.

### BRIEF DESCRIPTION OF THE FIGURES

FIGS. 1 to 22 show embodiments of the present invention.

FIG. 23 A shows CRL-1918 spheroids without the use of the Taxus Liberté stent. FIG. 23 B shows CRL-1918 spheroids after use of Taxus Liberté stent.

FIG. 24 shows an implant comprising three onion-like device bodies in tandem positions.

FIG. 25 shows an implant comprising a pearl-chain-like device body with differential pearl size diameters.

FIG. 26 shows an implant comprising a pearl-chain-like device body with tandem filters with different membrane locations.

FIGS. 27 A and B show different orientations of a filter membrane vis-à-vis the longitudinal axis of the implant body.

FIG. 28 shows an implant wherein cross-sectional planes of the circular loops or ellipsoids are oriented parallel to each other or may be oriented in an angle to each other.

FIG. 29 shows a Filterwire (Boston Scientific) device comprising filters resembling umbrellas on a wire with uniform pores of 80 μm-110 μm in diameter.

FIG. 30 shows an implant comprising a retrievable embolic filter.

FIG. 31 shows a bioactive coated implant comprising a microfilament assembly. The assembly is shown in 3 different versions, i.e. an open, closed and spiral version (1, 2 and 3). The figure further indicates the application of the catheter for placement and retrieval of the assembly.

FIG. 32 depicts the human artery system.

FIGS. 33 to 35 show examples of clinical uses of the filter device assembly implant according to the present invention.

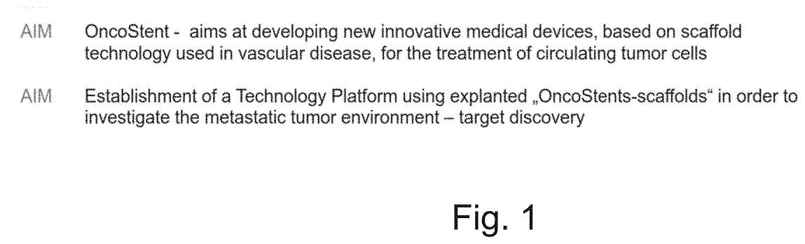

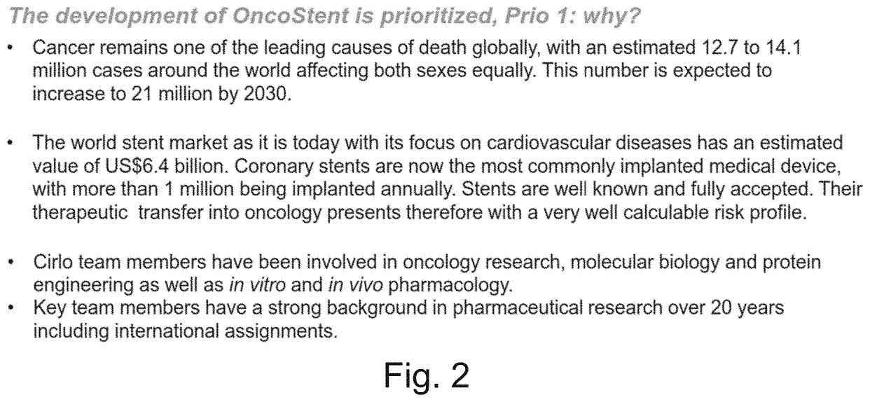

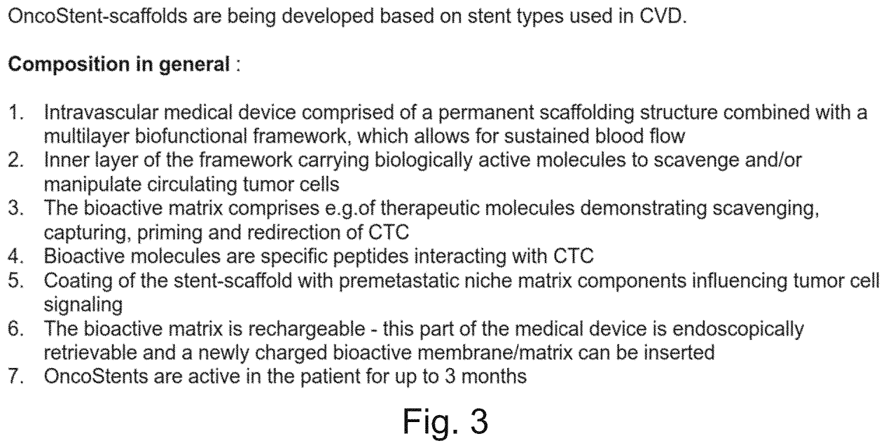

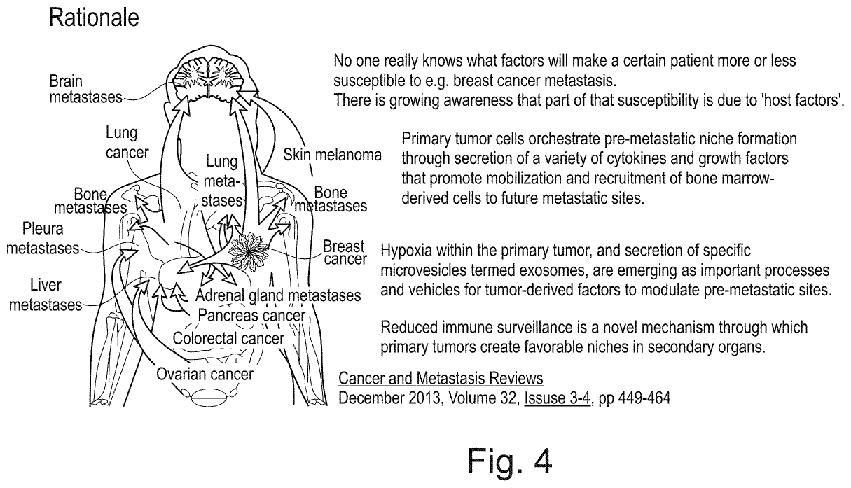

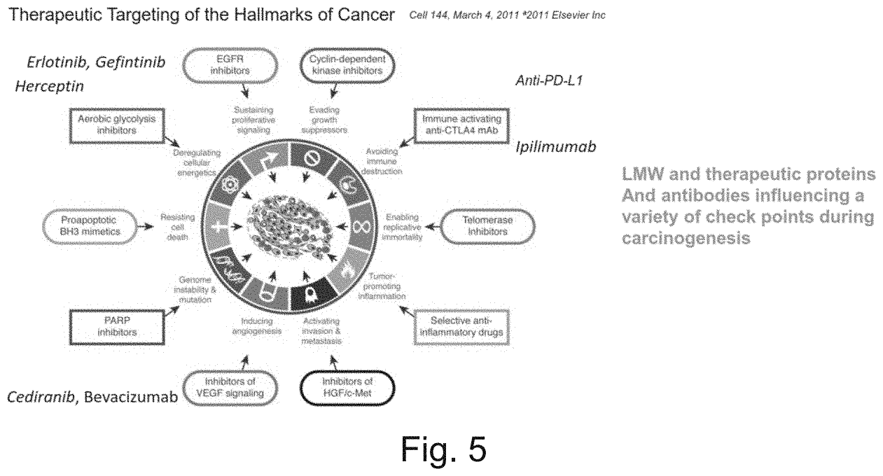

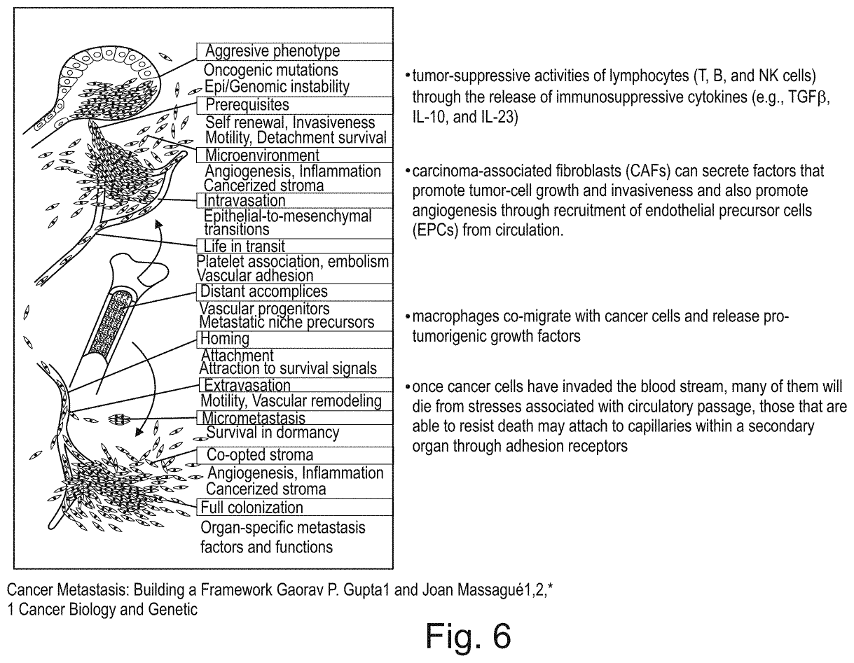

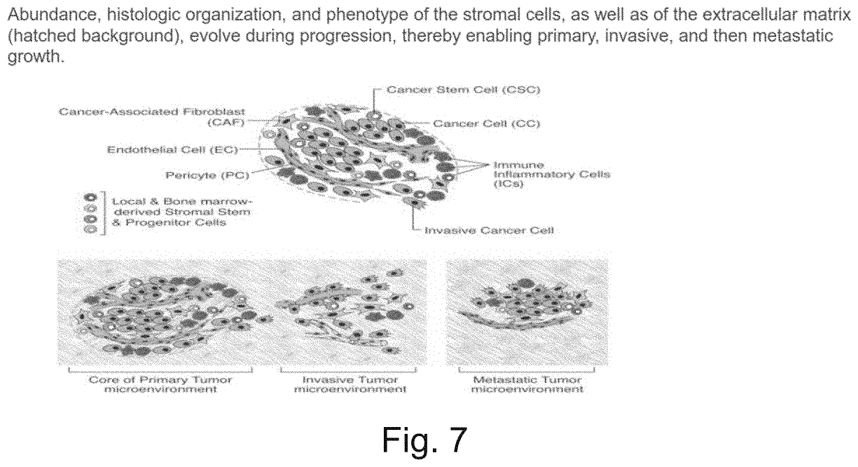

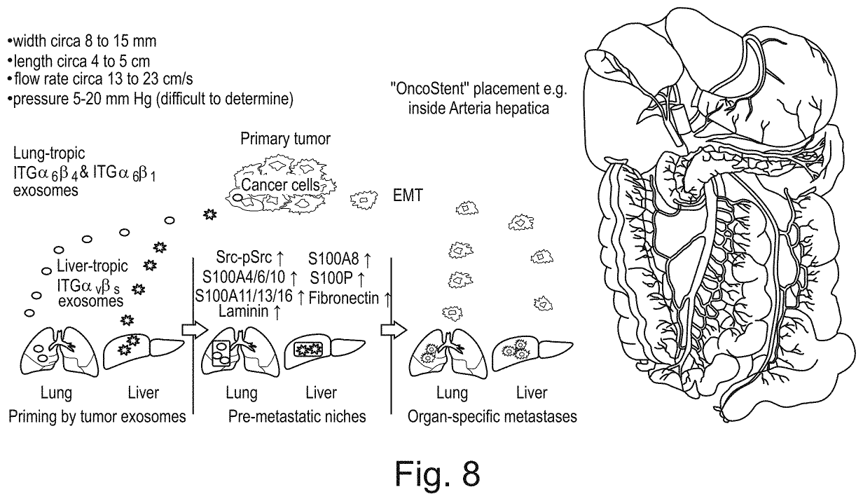

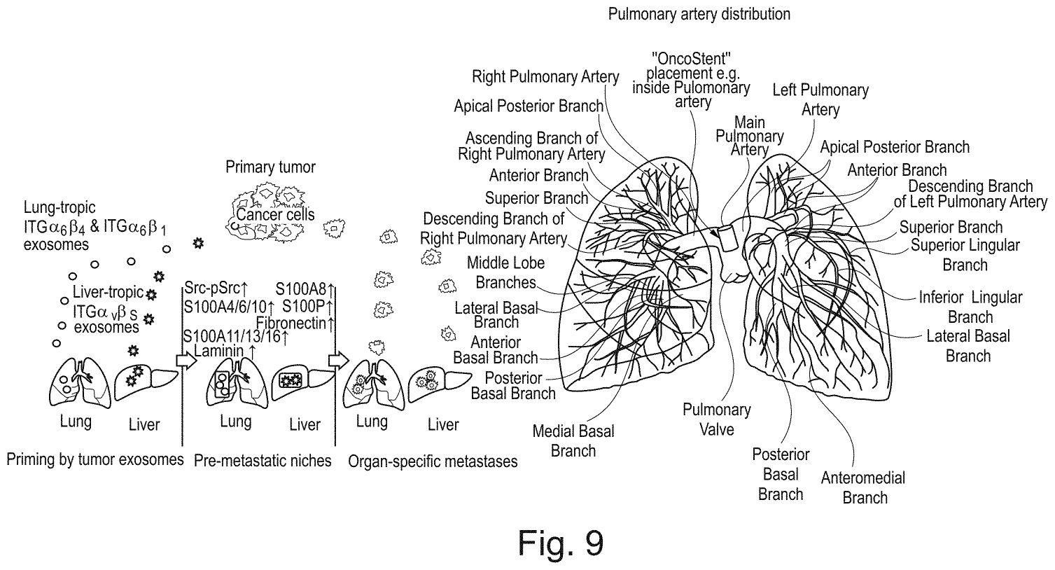

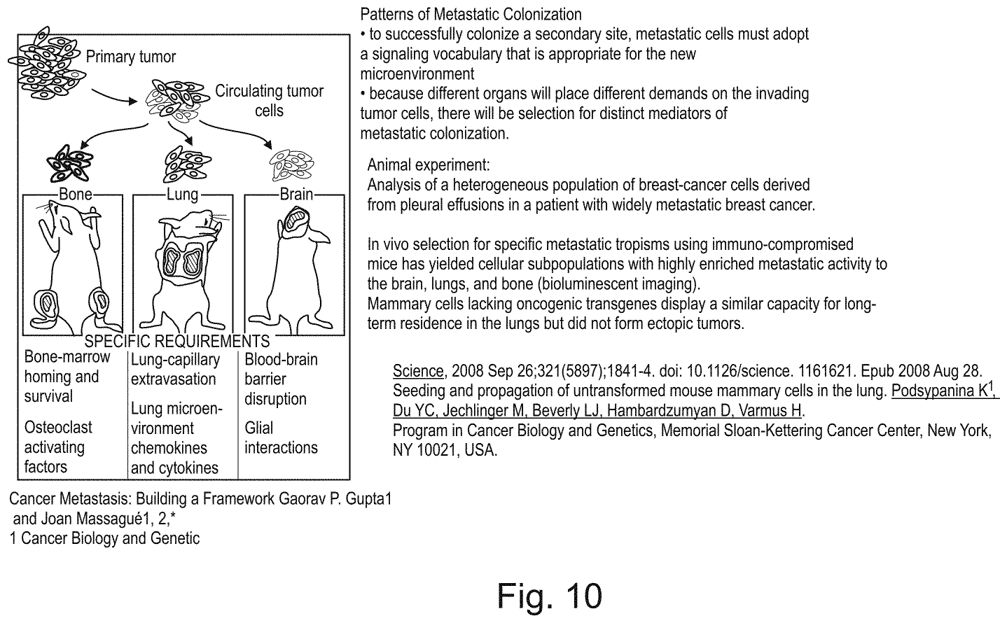

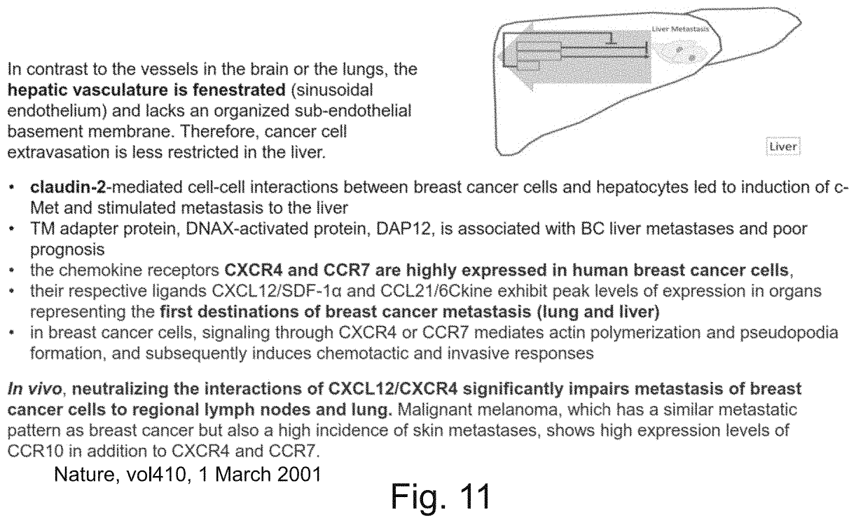

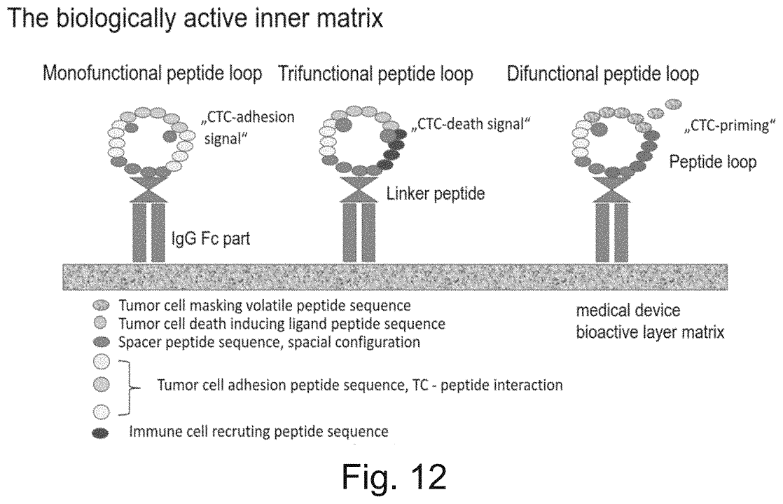

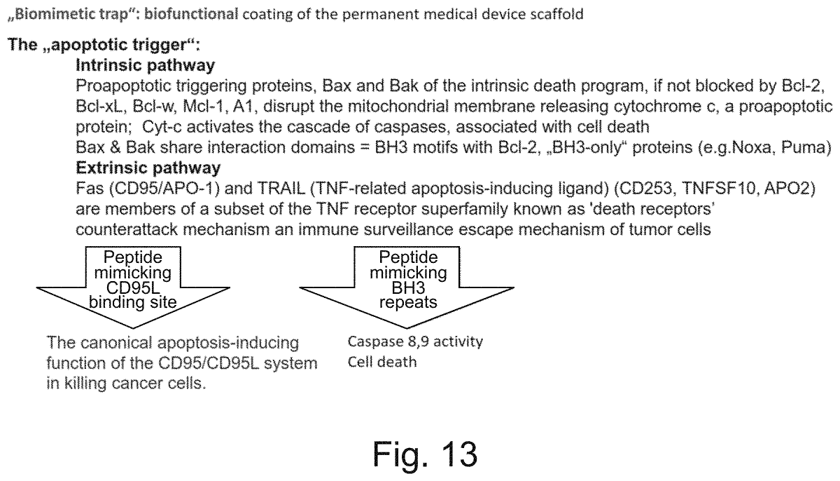

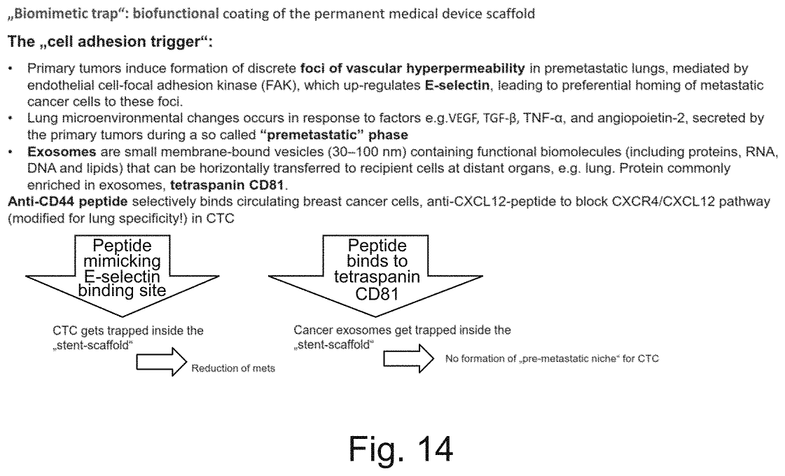

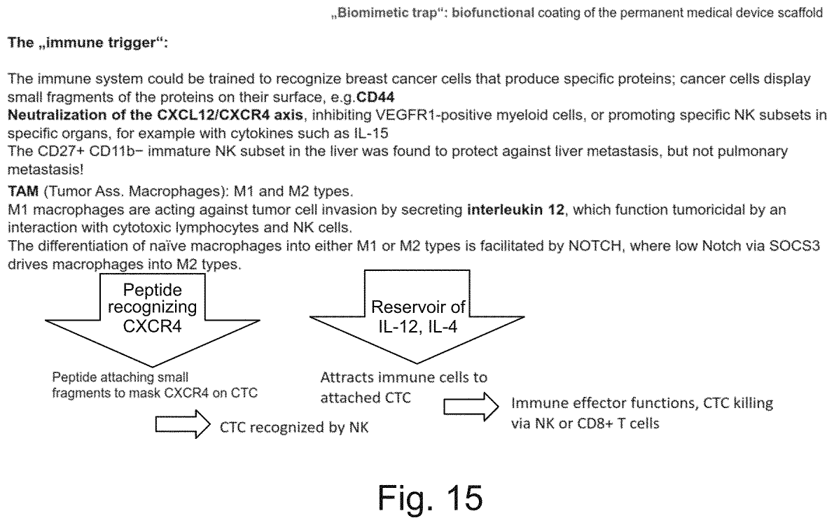

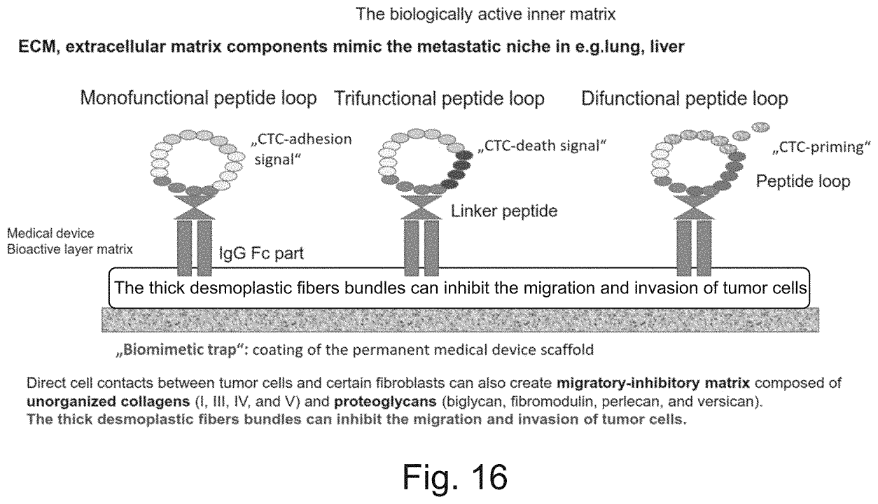

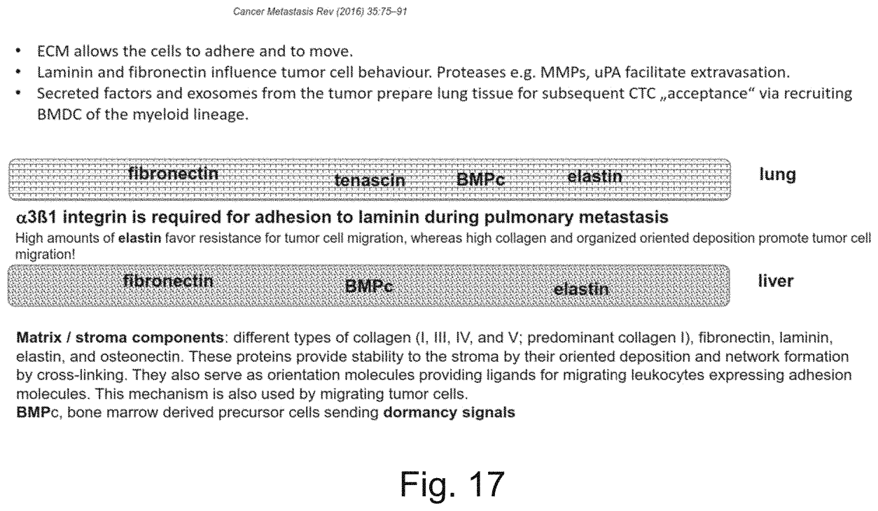

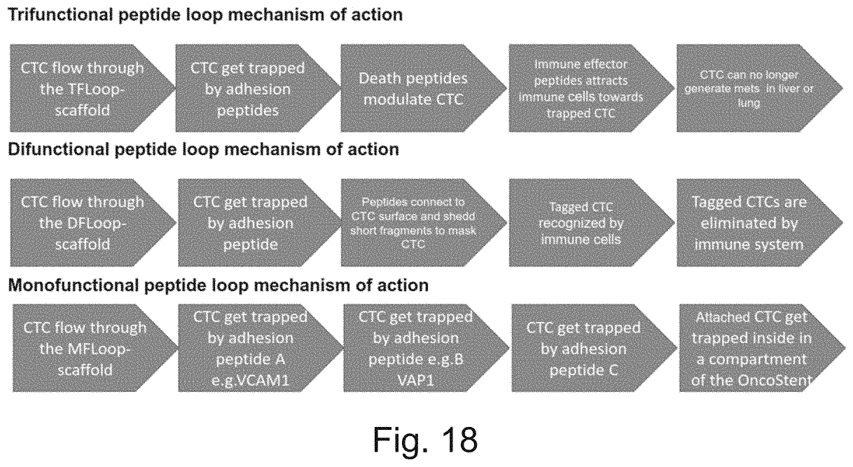

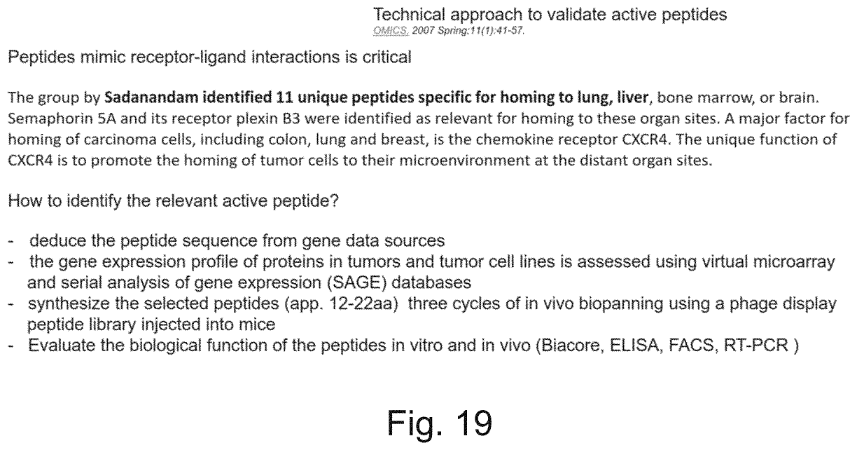

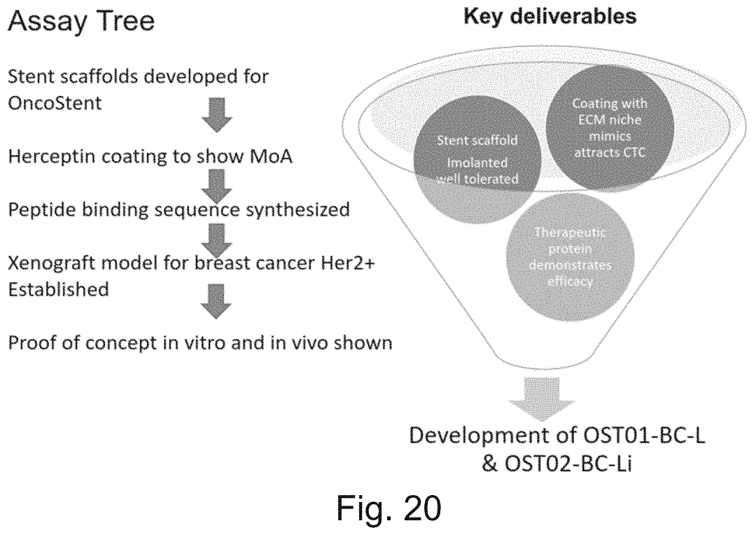

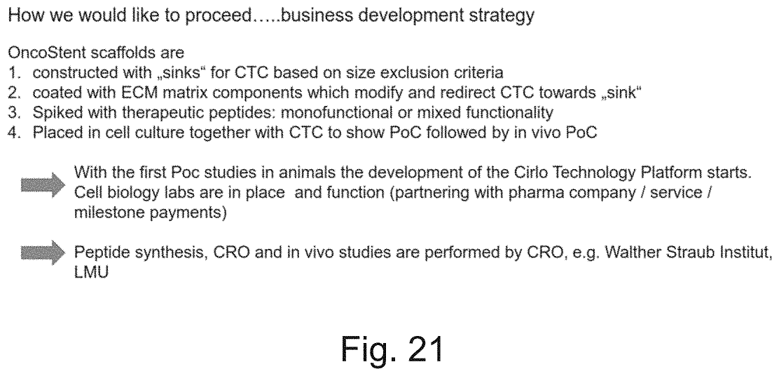

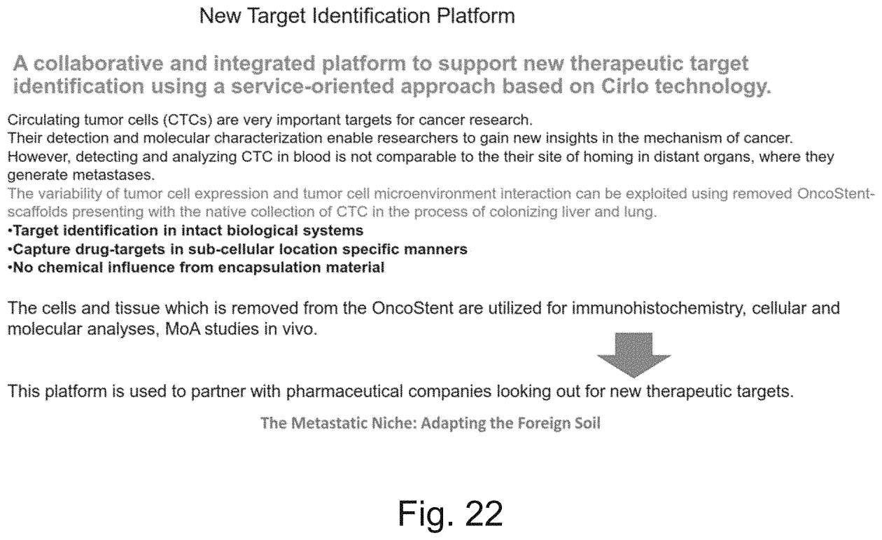

### DETAILED DESCRIPTION OF THE INVENTION

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” or “essentially consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “(i)”, “(ii)”, “(iii)” or “(a)”, “(b)”, “(c)”, “(d)”, or “first”, “second”, “third” etc. and the like in the description or in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms relate to steps of a method or use there is no time or time interval coherence between the steps, i.e. the steps may be carried out simultaneously or there may be time intervals of seconds, minutes, hours, days, weeks etc. between such steps, unless otherwise indicated.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As has been set out above, the present invention concerns in one aspect a filter device assembly implant comprising one or more chemical and/or biological agents wherein the implant is capable of recruiting a circulating tumor cell or a circulating metastatic cell or motile parts of tumor cells and thereby removes said cell or motile part thereof from circulation.

The term “filter device assembly implant” as used herein relates to a device which is designed to be implanted into a mammalian body, preferably into a human body. The device comprises a filter function for circulating cells in the blood circulation. In a typical embodiment, the device is capable of filtering and thereby recruiting circulating tumor or circulating metastatic cell or motile parts of tumor cells. The filtering and recruiting activity is designed to lead to a removal of circulating tumor and/or circulating metastatic cell and/or motile parts of tumor cells, as well as cell aggregates formed by tumor or metastatic cells, from bodily fluids. It is preferred that said removal is from the bloodstream or from lymphatic fluids. The present invention, in particular envisages the recruiting of cells, as well as of motile parts of tumor cells such as circulating microvesicles which are small membrane-bound cell fragments (sizes between 30 and 1000 nm diameter) or exosomes, which have 30 to 100 nm in diameter. Circulating microvesicles and exosomes were recently shown to have roles in cell signaling and intercellular molecular communication. Circulating microvesicles are typically actively released into the extracellular space to interact with specific target cells and have been demonstrated to deliver bioactive molecules. In many tumors, circulating microvesicle levels increase. The level and biochemistry of exosomes and microvesicles may provide suitable indicators for tumor severity.

The filtering and recruiting activity of the device is mainly achieved by the presence of one or more chemical and/or biological agents which are comprised on said device and fulfil their activity in situ. An accessory effect improving the filtering or recruiting may be provided by the form and design of the device itself, e.g. by reducing the circulation velocity of the bodily fluid, preferably of the blood stream. Accordingly, the retention time of circulating tumor cell and/or circulating metastatic cell and/or motile parts of tumor cells at or on the device can advantageously be extended, allowing for molecular interactions between the circulating tumor cell and/or circulating metastatic cell and/or motile parts of tumor cells on the one hand, and the one or more chemical and/or biological agents located on the device on the other hand.

The filter device assembly implant according to the present invention may be provided, in preferred embodiments, with one or more suitable properties, which can be combined or mixed according to necessities or circumstances. In certain embodiments, all properties may be given in one filter device assembly implant. The device according to the invention is hence designed to comprise one or more of said properties, in particular of properties (i) to (v) as mentioned below.

These properties include: (i) the device is catheter based. The term “catheter based” as used herein means that the device can be packed and/or provided to the patient inside of a catheter. The term “catheter” as used herein relates to a thin tube made from medical grade materials that can be inserted in the body to treat diseases or perform a surgical procedure. A catheter may be any suitable catheter known to the skilled person. Typical examples include polymers based catheters comprising material such as silicone rubber, nylon, polyurethane, polyethylene terephthalate (PET), latex, or thermoplastic elastomers. Also envisaged are polyimidine catheters. The catheter may, in certain embodiments, be connected to a deployment mechanism and may house a medical device that can be delivered over a guidewire. The catheter may include a guidewire lumen for over-the-wire guidance and may be used for delivering a device according to the invention to the target vessel. In certain embodiments, the catheter may have braided metal strands within the catheter wall to increase structural integrity. The structural elements of the catheter tip may further be bonded or laser welded to the braided strands of the catheter to improve the performance characteristics of the catheter tip.

Furthermore, (ii) the filter device assembly implant is freely positionable in a target vessel. The term “freely positionable” means that the device can be placed at any position in a target vessel. Potential size differences between target vessels may be reflected by using differently sized devices, or by making use of mechanisms which allow to adjust the size of the device to the target vessel, e.g. by further opening or closing the device. It is preferred that device is designed to be freely positionable in a minimal invasive surgery approach, e.g. with endoscopic technology including cameras and grapplers etc.

Furthermore, (iii) the device is retrievable. The term “retrievable” as used herein means that the device can removed from its implantation site, e.g. by reducing its size or retracting extended elements, without leaving significant residues and without destroying or damaging the target vessel where is has been implanted. The way the device is retrieved can be any mechanism known to the skilled person. Preferably, the retrieval is performed by catheter means. It is further preferred that the retrieval be performed in a minimal invasive manner, e.g. making use of catheters, endoscopic technology etc. It is particularly preferred that least one docking element is present at the proximal end of the device. The term “docking element” as used herein relates to a structural component which allows for an interaction with an auxiliary tool such as a catheter or endoscope tool etc. The interaction may, in particular, be designed for a retrieval of the device, e.g. after a certain period of time. The docking may, for example, include a mechanical coupling between the device and an interaction tool such as a catheter. Alternatively, the device may provide a docking element in the form of a protrusion which is easily reachable and detectable, allowing for a grabbing or catching of the device, e.g. in analogy to vena cava filters.

A further property of the device according to the present invention is that it is anchorable in a target vessel. The term “anchorable” as used herein means that the device cannot be moved or does not float within the target vessel where it has been implanted, but stays at the position of its implantation. This is preferably achieved by a contact between the expanded device and the surrounding tissue or vessel wall wherein the contact force is depending on the radial expansion force of the device or certain device elements, e.g. in analogy to self-expanding peripheral stents or vessel occluders. Also anchoring can be achieved by adding extensions of the device which seek contact or extrude into neighboring tissue or vessel walls as described for atrial occluders such as watchman occluders.

Yet another property of the device according to the present invention is that it is designed to fit into a permanent implant present in a target vessel. The device may, according to this property, be designed as a moveable and retrievable part of an implant. The implant may be present at a certain position in a target vessel. The device according to the present invention may be introducible into said implant and, e.g. after a certain period of time or after having reached the end of its working period (e.g. after 2, 3, 4, 5, 6, 7, 8, 9, 10, 11, 12, 15, 18, 20, 24 or more months), be removed therefrom. The device according to the invention may hence, in a preferred embodiment, be designed as a shuttle entity, which temporarily docks at a permanent implant present in a target vessel. The shuttle may, in further embodiments, be retrievable with a catheter, more preferably in a minimal invasive manner.

It is particularly preferred that the filter device assembly implant as defined herein be designed according to current implant techniques and technologies (e.g. stent implantation, septal occluder implantation, vena cava filter implantation). It is further preferred that it comprises an inactive (unexpanded, folded or constraint state) and an active (expanded, unfolded, unconstraint) state. The device is introduced into the target organ in an unexpanded state and is released at the target position into the expanded and active state

In certain embodiments the filter device assembly implant according to the invention comprises a reversible expandable device body having a proximal and a distal end. The device may accordingly be expanded along the line from the proximal to the distal end. The expansion may be directed in along the line from the proximal to the distal end, or perpendicularly thereto. The expansion may, in specific embodiments, increase the volume of the device by 10, 20, 30, 40, 50, 60, 70, 80, 90, 100, 150, 200, 300, 400, 500, 600, 700, 800, 900, 1000% or more than 1000% or by any value in between the mentioned values. It is also envisaged by the present invention that the device as defined above comprises at least one filter membrane. The term “filter membrane” as used herein relates to a selective barrier, mainly performing the function of a separator, e.g. by allowing for a filtering process as describe. The filter membrane may, accordingly, be designed to allow for partial separation of ingredients of bodily fluids such as blood or lymphatic fluid. Preferably, the filter membrane may be designed to allow for the passage of liquid portions, proteins or subcellular fragments in bodily fluids, e.g. blood. It is further preferred that blood cells like erythrozytes, thrombozytes can pass the membrane. It is further particularly preferred that the filter membrane is incapable of allowing the passage of cellular entities such as circulating tumor cells, circulating metastatic cell, cell aggregates of tumor cells or metastatic cells. In certain specific embodiments also motile parts of tumor cells, e.g. exosomes originating from primary tumors may not pass the membrane. Advantageously, the filter device assembly implant is thus capable of providing an increase, preferably maximized exposure of the membrane surface to the bodily fluid, e.g. the blood or lymph circulation, while at the same time avoiding stasis or thromboses. Accordingly, a maximized filter surface is provided allowing for an maximized blood exposure.

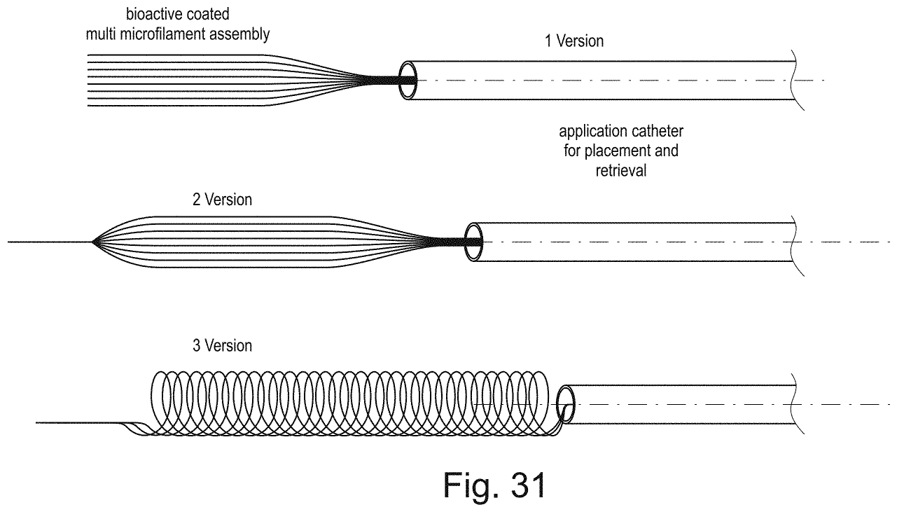

In a further set of embodiments, the filter membrane comprises, essentially consists of, or consists of one or more microfilaments. For example, the filter membrane comprises, essentially consists of, or consists of a multitude of longitudinally extending microfilaments, each of which is coatable by bioactive agent, thus creating a large bioactive surface area and permitting blood flow. The microfilaments may, in certain embodiments, be arranged parallel and straight or parallel and spiraling or in other arrangements. Further details may also be derived from FIG. 31, which shows and illustrates the corresponding embodiment.

In preferred embodiments, the filter membrane may comprise pores. In preferred embodiments, these pores may have a diameter of about 7 μm to about 100 μm. For example, the pores may have a diameter of 7, 8, 9, 10, 15, 20, 30, 40, 50, 60, 70, 80, 90 or 100 μm or any value in between the mentioned values. The pores may, in further embodiments, be provided with the same diameter, or with two or more different diameters. It is preferred that pores with different diameters be present.

In embodiments, in which more than one filter membrane is present in a device, said filter membranes may preferably have differential pore diameters and/or differential pore pattern. It is particularly preferred that the diameter ranges from about 7 μm to about 100 μm, e.g. has a value of 7, 8, 9, 10, 15, 20, 30, 40, 50, 60, 70, 80, 90 or 100 μm or any value in between the mentioned values. The term “differential pore pattern” as used herein refers to the geometric arrangement of groups of pores on the filter membrane, e.g. 4 to 100 pores, or groups of such basic groups pores etc. This arrangement may have any suitable form, e.g. be an equidistant pattern, or have a circular, rectangular, ellipsoid, linear, concentric or stellar form. The mentioned pattern may be different over the extension of one filter membrane, e.g. a proximal circular pattern may be followed by a more distal rectangular pattern etc. In case more than one filter membrane is present in a device according to the present invention, the pattern between said more than one filter membranes may be different, e.g. one filter membrane may show a circular pattern, whereas the neighboring filter membrane may provide a rectangular pattern etc.

The filter membrane may, in specific embodiments, be itself expandable. Accordingly, an expansion of the device may be followed or implemented by an expansion of the filter membrane. In certain embodiments, the filter membrane is attached to or arranged with the proximal and distal end of the filter device assembly implant. In further embodiments, the filter membrane is designed as a non-permanent, retrievable filter membrane. According to these embodiments, the filter membrane may be separated from the remainder of the device and be removed therefrom, e.g. with a catheter or based on endoscopic techniques. The filter membrane may, in further embodiments, be designed as replaceable entity allowing for an exchange of a filter membrane after a certain period of time or after having reached the end of its working period (e.g. after 2, 3, 4, 5, 6, 7, 8, 9, 10, 11, 12, 15, 18, 20, 24 days; after 2, 3, 4, 5, 6, 7, 8, 9, 10, 11, 12, 15, 18, 20, 24 weeks; or after 2, 3, 4, 5, 6, 7, 8, 9, 10, 11, 12, 15, 18, 20, 24 or more months). It is preferred that the end of the working period does not surpass 24 months. In specific embodiments, the filter membrane may be retrievable as whole, or in parts. The partial retrievability may be implemented as segmented retrievablity, e.g. via separable portions of the filter membrane, e.g. 10%, 20%, 30%, 40%, 50%, 60%, 70%, 80%, 90% etc., which can individually be retrieved, e.g. the distal part may be retrieved, whereas the proximal part is not retrieved etc. In further embodiments, any retrieval may followed by replacement of the retrieved filter membrane by a new filter membrane or the same filter membrane after washing and preparation for a secondary use. Also envisaged is the retrieval of more than one segment at a time.

The filter membrane may be composed of any suitable material. Envisaged examples include elastic or foldable polymer materials. Particularly preferred is polyurethane. Also envisaged is the use of micromeshes. In preferred embodiments, these micromeshes may comprise ultrathin wires, metallic or polymeric materials. In particularly preferred embodiments, the filter membranes according to the present invention is at least partially coated with one or more chemical and/or biological agents as defined herein above or below. The term “coated” as used herein means that all or at least some sectors of the filter membrane material as defined herein is covered by said chemical and/or biological agent(s). The coverage may, in certain embodiments, be present at one or both sides of the filter membrane. The “side” of the filter membrane refers to the geometric from of said filter membrane which has the shape of a thin sheet, thus comprising two sides, while the edge is not counted as side. In preferred embodiments, the filter membrane may be fully or partially coated on its interior side only. The “interior side” as used herein refers to the side of the filter membrane which is proximal to the lumen of the device or distal to the vessel wall, where the device is anchored. In further embodiments, the filter membrane may be fully or partially coated on its exterior side only. The “exterior side” as used herein refers to the side of the filter membrane which is distal to the lumen of the device or proximal to the vessel wall, where the device is anchored. In yet another set of embodiments, the filter membrane may be fully or partially coated on both sides. A “partial coating” may be a coating of about less than 1% to about 99% of the area of a filter membrane, e.g. of one side of the filter membrane or both sides of the filter membrane as defined above. For example, a partial coating may comprise 1, 5, 10, 15, 20, 25, 30, 40, 50, 60, 70, 75, 80, 85, 90, 91, 92, 93, 94, 95, 96, 97, 98 or 99% or any value in between the mentioned values of the area of a filter membrane.

In certain embodiments, the coating may differ between different filter membranes of a device. For a device comprising 2, 3, 4, 5, 6, 7 or more different filter membranes, a number of 2, 3, 4, 5, 6, 7 or more coatings may be provided. Alternatively, different filter membranes may be provided with one coating only. In further embodiments, the coating may change along the axis of the device, e.g. from proximal to distal over one filter membrane or over various filter membranes. In further embodiments, biological agents may be provided as coating in different combinations, e.g. as combination of adhesion and death signals, or as a combination of adhesion and immune modulation function.

In further embodiments the filter membrane as defined herein covers at least one cross-sectional area of the body of a device according to the present invention, in particular of the body of a device having a tubular form. For example, the filter membrane may cover 1, 2, 3, 4, 5, 6, 7, 8, 9, 10 or more cross-sectional areas of said body of a device according to the present invention. It is particularly preferred that the plane of the filter membrane is arranged perpendicular to the direction of the longitudinal axis of the body of a device according to the present invention. Also envisaged are different angles, e.g. non-perpendicular angles, between the plane of the filter membrane and the direction of the longitudinal axis, e.g. 10°, 20°, 30°, 40°, 50°, 60°, 70°, 80° or 85° or any value in between said values.

It is further envisaged that the filter device assembly implant according to the present invention comprises at least two filter membranes, each of which incompletely covers the cross-sectional area. These filter membranes may be arranged in any suitable form or shape. It is preferred that the filter membranes are arranged in tandem position along the longitudinal axis of the device body. Particularly preferred is a tandem arrangement along the longitudinal axis of the device body, wherein said filter membranes are opposite to each other within the circumference of the device body. Alternatively, they may be shifted in clockwise orientation. Such a shifting may advantageously be implemented for more than 2 filter membranes in tandem position, e.g. 3, 4, 5, 6, 7, 8, 9, 10 or more filter membranes. The filter device assembly implant according to the present invention may, in further embodiments, comprise alternating non-completely covering filter membranes as defined herein

The present invention also envisages that the filter membrane is self-expandable. In further embodiments, the entire filter device assembly implant is self-expandable. The term “self-expandable” as used herein relates to the property of the filter membrane or device to be expandable within a target vessel once it has reached its envisaged destination. The self-expansion may, for example, be started when withdrawing auxiliary tools such as catheters, as defined herein above. The self-expansion of the filter membrane or entire device may, in preferred embodiments, be activated by balloon inflation. It is preferred that at least a portion of the device, or the entire device be activated by balloon inflation. The term “balloon inflation” as used herein refers to the activity of expansion of a device by inflating a balloon element within the device. For example, a self-expanding device may comprise securing bands preventing the self-expansion of the device during the passage to the extended destination. Upon arrival, a balloon may be introduced into the device and be inflated within the device. Subsequently, said securing bands may be broken which leads to a self-expansion of the device. Subsequently, the balloon may be withdrawn, e.g. via catheter element.

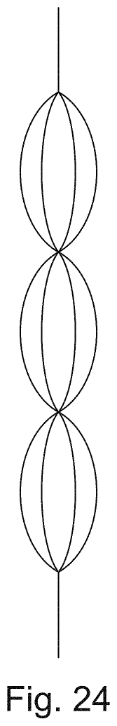

The present invention further envisages that the filter device assembly implant is provided in any suitable form or architecture. It is particularly preferred that the filter device assembly implant is provided in a tubular, onion like, pearl-chain-like, or a birds-nest like shape, or in any mixture of these shapes. Examples of corresponding forms and shapes can, for example, be derived from FIG. 24, 25, 26, 27, 28, 29, 30, or 31. The form or shape of the device may, in certain embodiments, be followed also be the form or shape of the filter membranes. For example, the filter device assembly implant according to the present invention may comprise alternating non-completely covering filter membranes which are provided in a pearl-chain, onion type or birds-nest like shape. In other embodiments, the filter device assembly implant comprises a multitude of longitudinally extending microfilaments, which may be arranged parallel or non parallel to each other in straight configuration or tortuous e.g. spiraling configurations.

In a particularly preferred embodiment, the filter device assembly implant as defined herein additionally comprises a retrievable embolic filter. The term “embolic filter” as used herein relates to a basket like structure at the distal end of the device, which is capable of catching debris or clots from the bodily fluid, e.g. from the blood, thereby preventing embolic events. The embolic filter has a preferably pore diameter of >100 μm, e.g. 110, 120, 130, 150, 200, 300, 400 or 500 μm or more or any value in between the mentioned values. Further details would be known to the skilled person or can be derived from suitable literature sources such as Shashni et al., Biol Pharm Bull, 2018; 41(4):487-503.

The device may be composed of, or be partially composed of, or comprise any suitable structural support material. According to certain embodiments, the structural support material may be metal. Preferred examples are stainless steel, gold, titanium, gold-titanium alloy, cobalt-chromium alloy, tantalum, platinum-radium alloy, tantalum alloy, magnesium, nickel-titanium alloy, e.g. nitinol, silver or copper. Alternatively or additionally, the support material may be plastic or polymeric material. Also envisaged is the use of memory shape materials, e.g. elastic memory shape meshwork material. Preferred examples include memory shape elastic wires. In yet another alternative, the structural support material may be a material readable by tomography or other imaging techniques, e.g. X ray. The present invention further envisages that the device body is not biodegradable or composed of biomaterial or biodegradable material. The term “device body” as used herein relates to all parts of the filter device assembly implant besides the one or more chemical and/or biological agents as defined herein above. Typically, the device body relates to the structural components of the device which provide for suitable placement at the desired target vessel and its capability of resisting impacting surrounding forces such as blood flow, vessel contraction or the like.

Further envisaged is that the filter device assembly implant comprises a radiopaque marker. The term “radiopaque marker” as used herein relates to materials which block X-ray radiation. Examples of such markers include platinum, gold, tantalum, or stainless steel, or a radiopaque ink. It is particularly preferred that the radiopaque marker is included in the device design. For example, the marker may be located at the proximal and distal end, or on at least two opposite portions of the outermost structural element of the device body. Such a design can advantageously allow to judge radial expansion under medical imaging, e.g. online X-ray analysis, and thus improve device expansion and placing etc.

The present invention further envisages in very specific embodiments that, for example, a tubular shape of the filter device assembly implant is implemented by a memory shaped spiraling wire, which forms a tubular spiral. More preferably, the memory shaped spiraling wire may be modified into a single spiral-like wire structure, characterized by incomplete wire circles and an interdigiting structure.

In a central aspect of the present invention the one ore more chemical and/or biological agents as mentioned above perform different chemical, biological and/or biochemical activities. Said activities may ultimately contribute and facilitate an effective recruiting of circulating tumor cells, circulating metastatic cell or motile parts of tumor cells, thus allowing for a removal of said cells or parts of tumor cells from the bodily fluid, e.g. from the blood and at the same time allow for a prolonged and safe use of the filter device assembly implant within a subject, as well as the efficient and extensive removal of these cells and parts thereof from the subject, even after a prolonged time, e.g. after 2 to 30 days, 4 to 8 weeks, or 1 to 24 months. To achieve one or more, preferably all, of these functions, the one ore more chemical and/or biological agents are provided as coating on the device, in particular on the filter membrane of the device as defined herein above.

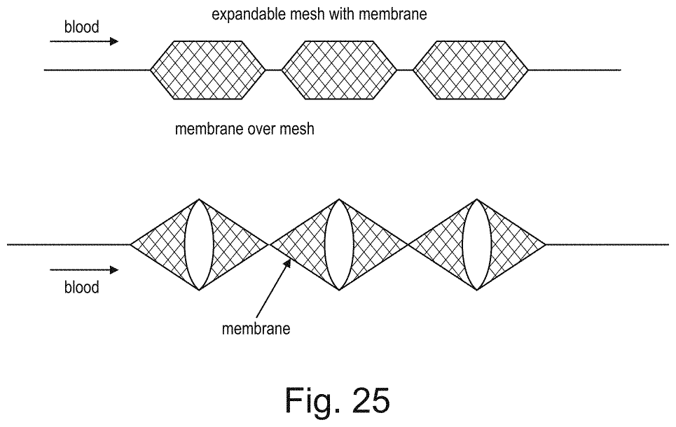

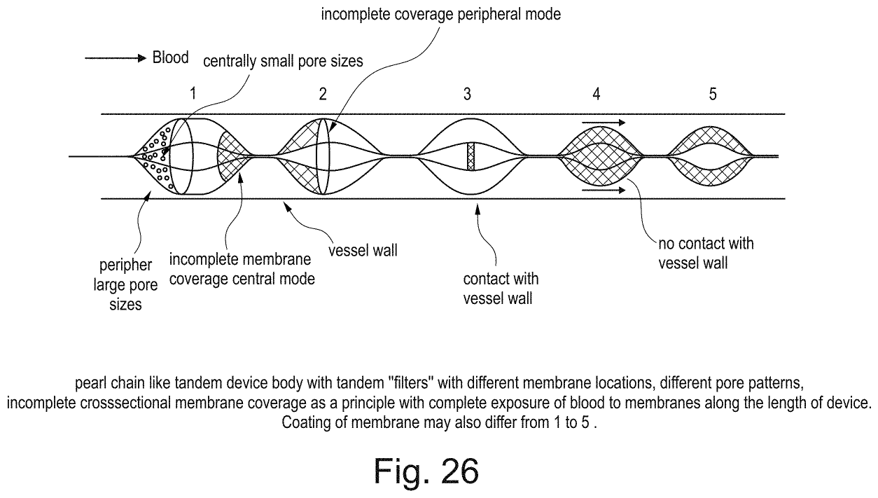

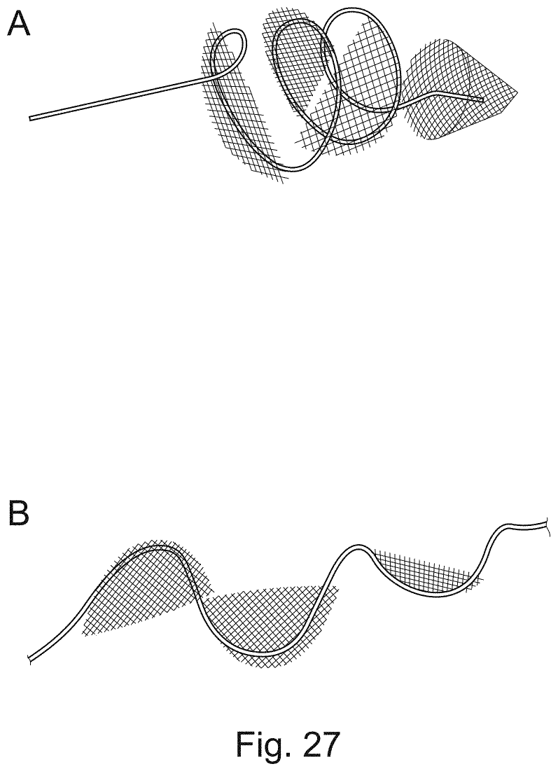

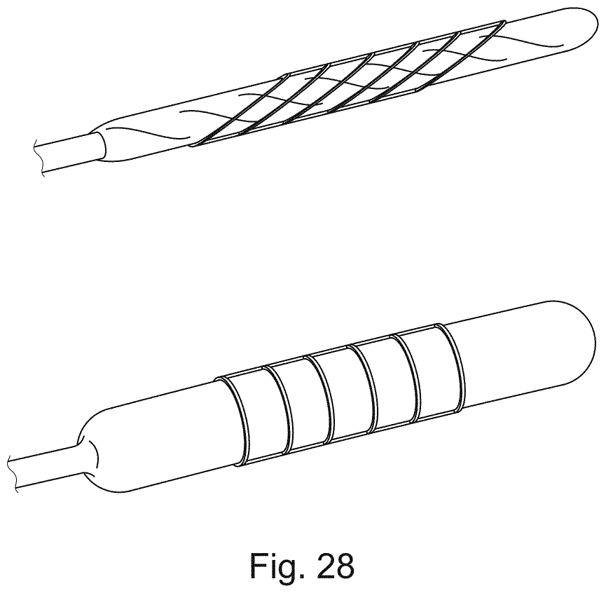

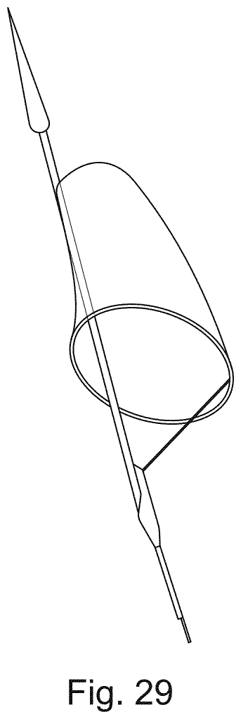

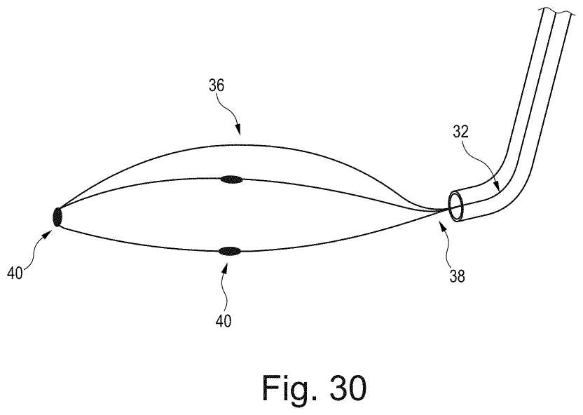

Without wishing to be bound by theory, it is assumed that such tumorous cell recruiting and removal processes at the surface of the filter device assembly implant are influenced by several factors, which may advantageously be used in the context of the present invention. One important factor, which is largely implemented by the physical form and design of the filter device assembly implant is the feeding of bodily fluids, e.g. blood or lymph, towards sections of the device which comprise one or more chemical and/or biological agents. For example, the filter device assembly implant may be designed such that filter membranes comprising said one or more chemical and/or biological agents are optimally exposed to bodily fluids, e.g. blood or lymph, comprising cells or parts thereof such as circulating tumor cells, for circulating metastatic cell or for motile parts of tumor cells. The exposure may further be improved by mechanical fluid manipulation, e.g. specific designs of the device such as depicted in FIGS. 24 to 31 or defined above. A further factor is the coating of the filter device assembly implant with suitable chemical or biological agents. The coating may fulfil different functions which can differ along the length of the device or according to its intended use. For example, the provision of a passive coating with one or more polymeric materials as defined herein below fulfils an antithrombotic function and may also serve as a basis for further chemical and/or biological coating.

A further important factor is the presence of an ECM-like structure on the surface of the device, as defined herein below. The ECM-like structure may be provided in an equal manner throughout the device, or it may be provided in certain sectors or regions or the device only, or in certain sectors or regions of the device different forms of these structures may be present. The ECM-like structure essentially fulfils the function of a structural and/or biochemical support for surrounding cells, which is assumed to be of elevated importance once cells have settled down on or within the filter device assembly implant. Furthermore, the ECM-like structure resembles the microenvironment of the tumor or metastases. This ECM-like structure which is typically composed of laminins, adhesion molecules or chemical entities serves as homing compartment within the device. Yet another important factor, which is considered relevant for the initial recruiting steps is the presence of a biological agent, which is capable of binding to a tumor marker on a cell or part thereof, in particular on a recruiting a circulating tumor or circulating metastatic cell or motile parts of tumor cells. These different factors may, in specific embodiments, present together at one area of the device, or they may fulfil their functions in different areas or the device. Also suitable mixture of any of these factors are envisaged. Furthermore, the present invention envisages different forms of filter device assembly implants which may comprise or implement either all or a sub-group of said factors. The overall shape, the intended use, the specific medical condition of a subject, the intended time of use and/or the tumor form may require an adjustment of the presence of above mentioned factors or functions. Additional factors which may also be taken into account are the tumor staging, i.e. the phase or stadium of the disease, and/or details on a pretreatment, e.g. whether the subject has already been treated, how successful this treatment was and the time period since the treatment, as well as a possible chemotherapeutic resistance etc.

Accordingly, a filter device assembly implant of the invention may, particularly preferred embodiments, comprise (i) a passive coating with one or more polymeric materials, (ii) as well as an ECM-like structure, and (iii) an active coating which is capable of binding to a tumor marker on a cell or part thereof. In further embodiments, at least one of these elements (i) to (iii) may not be present. The present invention envisages all combinations of (i), (ii) and (iii) as defined above.

In one group of embodiments of the invention the coating may thus be a passive coating. The term “passive coating” means that the coated agent is chemically or biochemically inert. Such a passive coating is typically not involved in cell recruiting or removal processes. Passive coatings may advantageously exhibit antithrombotic properties. Passive coatings may also serve as a basis for further chemical and/or biological coating. Passive coatings may also permit to design release kinetics of chemicals/drugs. A further function of the passive coating is protection against corrosion and/or the provision of linkage or conjugation option for additional coating layers, e.g. biological agents as defined herein. The passive coating may be performed, for instance, with one or more polymeric materials or may comprise such materials. Preferred exampled of such materials are ethylene vinyl acetate (EVA), latexes, urethanes, polyurethanes, polysiloxanes, styrene-ethylene/butylene styrene block copolymers (SEBS), polytetrafluoroethylene (PTFE) and linear aliphatic polyesters. The passive coating according to the present invention is connected to the filter device assembly implant as defined above via adherence mechanisms. In preferred embodiments, the adherence is conveyed via adhesive layer between the surface of the filter device assembly implant, comprising at the surface, for example, metal, such as stainless steel, gold, titanium, gold-titanium alloy, cobalt-chromium alloy, tantalum, platinum-radium alloy, tantalum alloy, magnesium, nickel-titanium alloy, e.g. nitinol, silver or copper; plastic or polymeric material; elastic memory shape meshwork material such as memory shape elastic wires or a material readable by tomography or other imaging techniques, e.g. X ray; or any mixture thereof, and said coating. The adhesive layer may, for example, be composed or may comprise sugar, starch, polyvinyl-alcohol or degradable products thereof.

In a further group of embodiments, the chemical and/or biological agents composing the coating of the filter device assembly implant constitute an extracellular matrix-like structure. The term “extracellular matrix like structure” or “ECM-like structure” as used in the context of the present invention relates to a structure which simulates the three-dimensional network of extracellular macromolecules of an ECM and thereby provides structural and/or biochemical support for surrounding cells. The ECM-like structure accordingly provides, at least partially, important functionalities for a recruiting and removal from circulation of circulating tumor cells, circulating metastatic cell or motile parts of tumor cells. The ECM-like structure, for example, is capable of slowing down cells, or may increase their interaction competence for binding interactions, e.g. with biological agents as defined herein. To provide an extracellular-matrix-like structure, the present invention envisages the provision of extracellular molecules typically present in the extracellular matrix, in particular in the extracellular matrix of a mammal, more preferably in the extracellular matrix of a human being. The identity of the ECM constituting macromolecules may be adapted to the subject in which the device is to be used. For example, for human beings the typical composition of human ECMs may be used. For animals, e.g. cats, dogs, horses, cattle etc. corresponding ECM compositions may be employed.

In specific embodiments, the macromolecules used to provide an ECM like structure, i.e. the macromolecules which are provided as coating for the device according to the present invention, are proteoglycans, non-proteoglycan-polysaccharides, elastin; fibronectin or laminin. Particularly preferred are mixtures of these macromolecules. Preferred examples of suitable proteoglycans include heparan sulfate, chondroitin sulfate and/or keratin sulfate. Preferred examples of suitable non-proteoglycan-polysaccharides include hyaluronic acid. The present invention further envisages the employment of ECM-like structures in the form of protein mixtures. Such mixtures are, for examples, secreted by Engelbreth-Holm-Swarm (EHS) mouse sarcoma cells. Also preferred are Matrigel, BioCoat or GelTrex mixtures.

According to the present invention, the filter device assembly implant comprising a coating as defined herein provides an environment for circulating tumor cells, for circulating metastatic cell or for motile parts of tumor cells. The environment may, for example, allow metastatic cells to settle down in or on the device and thus leave the bodily fluid circulation, e.g. lymph or blood. Similarly, tumor cells which are non-metastatic but circulate through the body may be stimulated to settle down in or on the device and thus leave the bodily fluid circulation, e.g. lymph or blood. This function is preferably implemented by the use of, or presence of a biological agent in or on the filter device assembly implant, in particular as part of the coating, wherein said biological agent is capable of binding to a tumor marker on a cell or part thereof, in particular on a recruiting a circulating tumor or circulating metastatic cell or motile parts of tumor cells. This type of biological agent which is capable of directly interacting with a tumor marker is referred to by the present invention as “active coating” or part of an active coating.

The term “tumor marker” as used herein is understood as a protein such as, for example, an enzyme, a structural protein, a receptor protein, or a hormone, a fragment of a protein, a conjugated protein, a peptide or a carbohydrate, which is present on the surface of a tumor cell or motile parts of tumor cells and/or is produced by these cells or parts thereof. Such tumor markers may be indicative for any type of tumor which leads to circulating tumor cells or to metastatic cells. Also envisaged are synthetic tumor markers, e.g. peptide based tumor marker based on artificial sequences known or assumed to relevant as tumor marker. It is preferred that the tumor markers are tumor markers specific for breast tumors, prostate tumors, pancreas tumors, colon tumors, small cell lung tumors, lymphoma, multiple lymphoma, T-cell tumors, Mycosis fungoides, Melanoma, neuroblastoma, sarcoma, fibrosarcoma, Wilms tumor or Squamous cell carcinoma. In further preferred embodiments, the tumor marker is CCR4, CCR6, CCR7, IGF, LFA-1, VLA-4, VLA-5, CD44, CD44 v4-v7, CD44 v6-v7, CD44 D3 (v6-v7), CD44-R (v8-v10), CD44 v10, CD-44R1, CXCR3, CXCR4, CXCR5, CXCR6, CXCR7, Surface Fibronectin, PECAM-1 (CD31), CAM 120/180, Integrin alphaᵥ beta₅, P-Selectin, L-Selectin, Integrin alphaᵥ beta₅, Integrin alpha₄ beta₇, Integrin alpha₂ beta₁, Integrin alpha₂ beta₃, Integrin alphaᵥ beta₃, Galectin-3, N-CAM, L-Selectin, LPAM-I (alpha₄ beta₂), CTLA, Integrin alpha₄ beta₁, Integrin alpha_{E} beta₇, CCR10, Axl/Mer, Anxa2-R or Desmoglein I (DG I). Further envisaged are E-selectin ligands such as HCELL, PSGL1, MUC1, as well as LGALS3BP. These markers are known to the skilled person. Further information may be derived from suitable literature references such as Aceto et al., Cell, 2014, 158, 5, 1110-1122 or databases such as Genecards (https://www.genecards.org; last visited on Jun. 26, 2019), or Genbank at NCBI (https://www.ncbi.nlm.nih.gov/genbank; last visited on Jun. 26, 2019).

The term “capable of binding to a tumor marker” as used herein means that the biological agent performs a molecular, non-covalent binding interaction with said tumor marker and/or further components on a cell or part thereof, which allows for an at least temporary fixation of the cell carrying the tumor marker at the place of the biological agent. Without wishing to be bound by theory, it is assumed that such a binding is mediated by binding capacity depending on signals inside of the passing cells. Thee signals typically result from the interaction with the specific biological agents as defined herein. They may include, for example, cell death signals, or immune modulation signals. The present invention envisages several components which are capable of binding to a tumor marker. Preferred examples are a tumor-marker specific antibody or a fragment thereof, the protein CD133 or a fragment or domain thereof, the protein VEGFR-1 or a fragment or domain thereof, a homing factor or a fragment or domain thereof; and a tumor-marker specific lectin or a fragment or domain thereof.

Examples of tumor-marker specific antibodies envisaged by the present invention include panitumab, matuzumab, nimotuzumab or derivatives thereof. Further envisaged are additional antibodies against EGFR family members, or ErbB2/HER2 family members. Further details would be known to the skilled person or can be derived from suitable literature sources such as Huang and Buchsbaum, Immunotherapy, 2009; 1(2): 223-239.

CD133 is pentaspan transmembrane glycoprotein. The protein typically localizes to membrane protrusions and is often expressed on adult stem cells, where it is thought to function in maintaining stem cell properties by suppressing differentiation. The protein consists of five transmembrane segments, with the first and second segments and the third and fourth segments connected by intracellular loops while the secand and third as well as fourth and fifth transmembrane segments are connected by extracellular loops. Further information may be derived from suitable literature sources such as Glumac and LeBeau, Clin Trans Med, 2018, 7, 18.

VEGFR-1 relates to Vascular endothelial growth factor receptor 1 which is encoded by the FLT1 gene in humans. The protein has been shown to interact with PLCG1 and vascular endothelial growth factor B (VEGF-B).

The term “homing factor” as used herein relates to cell adhesion molecules which typically interact with corresponding or cognate interactors such as adresssins on target tissues. For example, a hepatic microenvironment essentially determines tumor cell dormancy and metastatic outgrowth of pancreatic ductal adenocarcinoma. Further information may be derived from suitable literature sources such as Lenk et al., OncoImmunology, 2018, 7, 1.

The present invention, in preferred embodiments, envisages the use of one or more of the following homing factors: Osteopontin, Hyaluronate, CXCL12, CCL21, Dipeptidyl Dipeptidase IV, PECAM-1, Bone Sialoprotein, Peripheral Node Addressin (CD34), MADCAM-1, VCAM-1, Collagen type I, Fibronectin, Osteonectin, N-CAM, FGF Receptor, GlyCAM-1, ICAM-1, ICAM-2, ICAM-3, E-Selectin, E-Cadherin, HECA-452, CCL27, CXCL9 (Mig), SDF-1, CXCL16, GAS-6, Anxa2, T140 or CXCL10 (IP10).

In specific embodiments any combination of the above mentioned elements, e.g. homing factors, CD133, VEGFR-1 or antibody may be used. Also envisaged is the use of one type of element, e.g. one specific homing factor, in a specific area of the device, followed by a different type of element in the neighboring area etc.

The present invention further envisages that a biological agent as defined herein, e.g. a homing factor, an antibody etc. is linked to the passive coating of the filter device assembly implant via a spacer element. Also envisaged is the linkage to the structural support material of the filter device assembly implant, e.g. stainless steel, gold, titanium, gold-titanium alloy, cobalt-chromium alloy, tantalum, platinum-radium alloy, tantalum alloy, magnesium, nickel-titanium alloy, e.g. nitinol, silver or copper; plastic or polymeric material; elastic memory shape meshwork material such as memory shape elastic wires or a material readable by tomography or other imaging techniques, e.g. X ray; or any mixture thereof, which may be present on the surface of said filter device assembly implant. The term “linked” as used herein refers to a persistent connection between said biological agent and the device. The linkage may either be performed with the structural support material as defined above. In such an embodiment, the linkage may be implemented as binding to a metal ion resin, preferably ion-NTA or ion-agarose. Alternatively, the linkage may be performed with the passive coating of the filter device assembly implant. In corresponding embodiments, the linkage may be implemented as covalent binding between parts of the passive coating and the biological agent or the spacer element attached to it.

The term “spacer element” as used herein relates to a distance piece which is capable of spatially separating the biological agent form the surface of the filter device assembly implant. This separating allows for a sterically unhindered interaction of the biological agent with a component on a circulating tumor cells or the like. In preferred embodiments the spacer elements are composed or partially composed of a peptide or a polypeptide. It is particularly preferred that the Fc part of an antibody or multi-histidine tag be used. Also envisaged is the employment of a nucleic acid; a modified nucleic acid; or a polymer such as PEG, PLA, PVA, polyethylene or polypropylene. The spacer element may have any suitable length. In a preferred embodiment, the spacer element has a length of about 1 to 20 nm, e.g. 1, 2, 3, 4, 5, 6, 7, 8, 9, 10, 11, 12, 13, 14, 15, 16, 17, 18, 19 or 20 nm or any value in between the mentioned values. Also envisaged are shorter spacer elements in a length of 7 to 25 amino acids, e.g. 7, 8, 9, 10, 11, 12, 13, 14, 15, 16, 17, 18, 19, 20, 21, 22, 23, 24, 25 amino acids, or equivalents thereof.

In addition to its function as spatial separator between the biological agent and the surface of the filter device assembly implant, the spacer element further fulfils the function of a separator between biological agents as defined herein. The present invention accordingly envisages the provision of biological agents in a density which allows for an efficient binding of the biological agent to a tumor cell or part thereof. According to preferred embodiments, the spacer elements may be provided in a density of 2 to 500 per μm² on the surface of the filter device assembly implant. For example, a density of 2, 5, 10, 15, 20, 30, 40, 50, 60, 70, 80, 90, 100, 150, 200, 250, 300, 350, 400, 450 or 500 per μm² on the surface of the filter device assembly implant may be used.

The present invention further relates to biological agents—as part of the filter device assembly implant—which comprise, essentially consists of, or consists of a binding domain capable of binding to a tumor marker. The term “binding domain capable of binding to a tumor marker” as used herein relates to an amino acid sequence which comprises a non-covalent binding functionality for a tumor marker as mentioned herein above. Preferably, the binding domain is capable of binding to any of CCR4, CCR6, CCR7, IGF, LFA-1, VLA-4, VLA-5, CD44, CD44 v4-v7, CD44 v6-v7, CD44 D3 (v6-v7), CD44-R (v8-v10), CD44 v10, CD-44R1, CXCR3, CXCR4, CXCR5, CXCR6, CXCR7, Surface Fibronectin, PECAM-1 (CD31), CAM 120/180, Integrin alphaᵥ beta₅, P-Selectin, L-Selectin, Integrin alphaᵥ beta₅, Integrin alpha₄ beta₇, Integrin alpha₂ beta₁, Integrin alpha₂ beta₃, Integrin alphaᵥ beta₃, Galectin-3, N-CAM, L-Selectin, LPAM-I (alpha₄ beta₂), CTLA, Integrin alpha₄ beta₁, Integrin alpha_{E} beta₇, CCR10, Axl/Mer, Anxa2-R or Desmoglein I (DG I). The present invention also envisages binding domains for further tumor marker including those which have not yet been identified.

In preferred embodiments, the binding domain is a peptide or polypeptide molecule. The domain may have any suitable length. It is preferred that it has length of about 20 to about 250 amino acids. More preferably, the binding domain has a length of about 20 to about 120 amino acids. In further preferred embodiments, the binding domain has a length of about 20, 25, 30, 35, 40, 45, 50, 55, 60, 65, 70, 75, 80, 85, 90, 95, 100, 105, 110, 115 or 120 amino acids, or any value in between the mentioned values.

Information on suitable binding domains capable of binding to a tumor marker can be derived from literature resources or databases, e.g. from Partyka et al., Proteomics, 2012, 12(13), 2212-2220.

The binding domain capable of binding to a tumor marker according to the present invention may be provided in any suitable form. For example, the binding domain may be provided as part of a larger protein structure, wherein said larger protein structure may fulfil presenting or structural functions, thus improving the expose of the binding domain. Further envisaged is combination of more than one binding domain per biological agent unit. The term “biological agent unit” as used herein means that a biological agent may comprise one or more functions, but is attached to the filter device assembly implant in a single manner, e.g. via a linker or spacer element as defined herein. Accordingly, a biological agent unit may comprise 2, 3, 4, 5 or more binding domains and/or structural protein components. Also envisaged is the presence of intervening peptide elements within the unit between the domains. Also envisaged is the additional use a linker element between the biological agent and said spacer. The additional linker may have a length of 1, 2, 3, 4, 5, 6, 7, 8, 9, 10, 11, 12, 13, 14, 15, 16, 17, 18, 19 or 20 amino acids. The linkage between the biological agent and the spacer may further be implemented as covalent or non-covalent connection.

In further embodiments, the binding domain capable of binding to a tumor marker according to the present invention may be combined with one or more additional functional domains. Preferably, such a further functional domain is or comprises, partially comprises or consists of an apoptosis inducing factor or a functional domain of an apoptosis inducing factor capable of inducing apoptosis. Preferred examples of suitable apoptosis inducing factors are FasL/CD95L, TNF-alpha, APO3L and APO2L/TRAIL. The presence of such apoptosis inducing factors advantageously allows the filter device assembly implant of the present invention to not only attract tumor cells or metastatic cells, but also to subsequently induce a killing of these cells via apoptotic processes. The present invention further envisages the use of additional killing approaches based on molecular interactions between the surface of a cancer cell or metastatic cell and an interactor.

In specific embodiments of the present invention the domain capable of binding to a tumor marker and the domain capable of inducing apoptosis are provided as fused domains. Also envisaged is the alternative that the domains are linked via a linker element of about 1 to 20 amino acids length. For example, the linker element may be a peptide having a length of 1, 2, 3, 4, 5, 6, 7, 8, 9, 10, 11, 12, 13, 14, 15, 16, 17, 18, 19 or 20 amino acids.

In further specific embodiments of the present invention the biological agent is provided as linear or circular element or as an element composed of linear and circular parts. It is particularly preferred to provide the biological agent as a linear or circular or partially linear or circular peptide or polypeptide. Typical embodiments envisage that the circular biological agent has or is part of a structure comprising a loop or a loop and a stem. Also envisaged are linear structures, which are linked to a spacer element as defined herein. The loop structure or linear structure may comprise said preferably comprise the biological agent, e.g. the tumor binding domain or the apoptosis inducing domain, at an exposed position. Said exposition allows for an efficient interaction, e.g. leading to the binding to a tumor marker or a tumor cell, or the induction of apoptosis.

In further specific embodiments a biological agent as defined herein above comprises or is linked to one or more additional elements. For example, the biological agent may be linked to a sugar, to branched or unbranched multiple sugar structures, to alkynes or azides, to streptavidin, biotin, amines, carboxylic acids, active esters, epoxides or to aziridines.

In yet another specific group of embodiments, the filter device assembly implant according to the present invention comprises a pharmaceutical agent. For example the pharmaceutical agent may be provided as part of a coating as defined herein. The pharmaceutical agent may, in certain embodiments, improve or support one or more functions of the filter device assembly implant, e.g. the recruiting or removal function for tumor cells or metastatic cells. For example, the pharmaceutical agent may be a cytotoxic compound which kills recruited tumor cells or metastatic cells. In particularly preferred embodiment, the pharmaceutical agent is an antiproliferative agent or an anticoagulant. The provision of an antiproliferative agent on or in the filter device assembly implant allows for a reduction of cell growth, e.g. tumorous growth or metastatic growth in the filter device assembly implant. Furthermore, the antiproliferative agent is useful to reduce the growth of endothelial cells in the neighborhood of the device, thus preventing an overgrowth or occlusion of the device, or an embolism. The provision of an anticoagulant agent on or in the filter device assembly implant allows for the prevention of blood clotting inside the filter device assembly implant and also an occlusion of the device or an embolism. Preferred examples of suitable antiproliferative agents are paclitaxel or sirolimus. Preferred examples of suitable anticoagulants are reteplase or heparin.

The pharmaceutical agent may be provided in an amount which is adjusted to the intended period of use. In further embodiments, the pharmaceutical agent may be released in a time-controlled manner, e.g. in a steady concentration over the entire intended period of use. Suitable controlled release technologies are known to the skilled person or can be derived from suitable literature references such as Senst B, Basit H, Borger J. Drug Eluting Stent (DES) Compounds. [Updated 2019 Apr. 29]. In: StatPearls [Internet]. Treasure Island (FL): StatPearls Publishing; 2019 January-. Available from: https://www.ncbi.nlm.nih.gov/books/NBK537349/.

In a further aspect the present invention relates to a filter device assembly implant as defined herein for use in treating cancer and/or metastasis, or for use in preventing cancer and/or metastasis in a subject.

The term “subject” or “patient” used herein refers to a mammal. “Mammal” as used herein is intended to have the same meaning as commonly understood by one of ordinary skill in the art. Preferred mammals are primates, cows, sheep, goats, horses, dogs, cats, rabbits, rats, mice and the like. In particularly preferred embodiments, the subject is a human being. The term includes as treatment target group persons affected by a pathological condition. Also envisaged as treatment target group are healthy subjects or subjects showing no symptoms of a disease, preferably showing no symptoms of cancer or metastasis. Further envisaged as treatment target group are subjects being at risk of developing cancer and/or metastasis.

The term “administered” as used herein relates to the provision and possible maintenance of a therapeutically effective form of the filter device assembly implant at any suitable place in the subject's body. By “therapeutically effective form” is meant a dose or number of biological agents on or in the filter device assembly implant that produces the effects for which it is administered. The exact dose or number of biological agents will depend on the purpose of the treatment, and will be ascertainable by one skilled in the art using known techniques. As is known in the art and described herein, adjustments for localized usage, age, body weight, general health, sex, diet, time of administration/use, drug interaction and the severity of the condition may be necessary, and will be ascertainable with routine experimentation by those skilled in the art.

The filter device assembly implant of the present invention may be used in both human therapy and veterinary therapy, preferably in human therapy.

The filter device assembly implant described herein may be used or administered alone or in combination with other treatments. Combination treatments are envisioned for cancer immunotherapy, for example via co-administration of checkpoint inhibitors such as anti-CTLA-4 and anti-PD1 antibodies, or for chemotherapy, for example by co-administration of alkylating agents or DNA and RNA polymerase inhibitors. Further combination approaches may involve targeting cancer cells by mitochondrial mediated apoptosis, targeting cancer cells by ROS mediated apoptosis, targeting cancer cells by death receptor mediated apoptosis, targeting cancer cells by cell cycle mediated apoptosis, targeting cancer cells by regulating multiple signaling pathways and transcription factors. Also envisaged are additional targeting approaches for the STAT3 Pathway, the PI3K/AKT/mTOR Pathway, the MAPK/ERK (Ras-Raf-MEK-ERK) Pathway, the Wnt/β-Catenin Pathway, for Hypoxia-inducible Factor-1α (HIF-1α), for the COX-2/PGE2 pathway, and combinations with cytokines and chemotherapy. In specific embodiments the death receptor peptides to be used in this context may be of the following type: Apo3L/TWEAK and DR3 APo-3; Apo-2L/TRAIL and DR4/5; TNFalpha and TNF-R2 and R1; FasL or Fas/CD95.

The terms “treat” or “treatment”, unless otherwise indicated by context, refer to therapeutic treatment and/or prophylactic measures to prevent the outbreak or relapse of a cancer disease, wherein the objective is to inhibit or slow down (lessen) an undesired physiological condition. For purposes of this invention, beneficial or desired clinical results include, but are not limited to, alleviation of symptoms, diminishment of extent of a cancer disease, stabilized (i.e., not worsening) state of a cancer disease, delay or slowing of a cancer disease progression, amelioration or palliation of the cancer disease state, and remission (whether partial or total), whether detectable or undetectable. “Treatment” can also mean prolonging survival as compared to expected survival if not receiving treatment. Those in need of treatment include those already having the condition or disorder as well as those prone to have the condition or disorder. The term “prevention” as used herein relates to prophylactic measures in order to impede or avoid the affection by cancer or cancerous metastasis of a subject. The prevention may, in certain embodiments, include quantitative or almost quantitative capturing of cancer cells or metastatic cells and a subsequent destruction or removal of these cells from the circulation.

The treatment or prevention may further, in specific embodiments, involve a single administration or use of the filter device assembly implant as defined above, or multiple administrations or uses. A corresponding administration or usage scheme may be adjusted to the sex or weight of the patient, the disease, the general health status of the subject etc. For example, the administration or use scheme may contemplate a usage within the subject's body for one week, two weeks, 3, 4, 5, 6, 7, 8, 10, 11, 12 weeks, 4, 5, 6, 7, 8, 9, 10, 11, 12, 18, 24 or more months, or any time period in between the mentioned periods. These usage schemes can of course be adjusted or changed by the medical practitioner in accordance with the subject's reaction to the treatment/prevention and/or the course of the pathological condition.

The filter device assembly implant as defined above may accordingly be administered to the subject in as a controlled active, controlled release and toxic entities (tumor cells or metastatic cells) accumulating agent.

The administration or implantation of the filter device assembly implant may preferably be performed during and/or after the treatment of a subject with a therapeutic agent. The treatment of a subject as mentioned here is typically an anti-cancer treatment. The administration or implantation of the filter device assembly implant may, in further embodiments, be preferably performed during and/or after surgery removing a tumor load. It is particularly preferred that the administration scheme foresees an implantation immediately after said surgery or, in the alternative, as ultimate step of the surgery to capturing not removed cancer cells.

The term “cancer” as used herein relates to a pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms typically show partial or complete lack of structural organization and functional coordination with the normal tissue and most invade surrounding tissues, metastasize to several sites, and are likely to recur after attempted removal and to cause the death of the patient unless adequately treated. As used herein, the term “neoplasia” is used to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma skin cancer, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma. Also envisaged are further cancer forms known to the skilled person or derivable from suitable literature sources such as Paylopoulou et al., 2015, Oncol Rep., 33, 1, 3-18. The cancer may, in certain embodiments, be a refractory cancer. A cancer may be assumed to be residually present if a subject has undergone surgery as treatment for the cancer.

The term “metastasis” as used herein relates to a tumor's spread from an initial or primary site to a different or secondary site within the subject's body. A metastasis may be provoked by or circulating metastatic cells, which are have typically acquired the ability to penetrate the walls of lymphatic or blood vessels, after which they are able to circulate through the bloodstream to other sites and tissues in the body, re-penetrate the vessel or walls and continue to multiply, eventually forming another clinically detectable tumor. A metastasis is hence a secondary tumor form based on circulating cells.

It is particularly preferred that the cancer to be treated or prevented is a colon cancer, breast cancer, lung cancer, melanoma, esophageal cancer, prostate cancer, pancreatic cancer, ovarian cancer, myeloma, or a lymphoma such as ALL, CLL, or AML. It is further particularly preferred that the metastasis to be treated or prevented is derived from a colon tumor, breast tumor, lung tumor, e.g. small cell lung tumors, squamous cell carcinoma melanoma, prostate tumor, pancreas tumor, lymphoma, T-cell tumor such as Mycosis fungoides, neuroblastoma, sarcoma, fibrosarcoma, ovarial tumor or nephroblastom such as Wilms tumor. Due to its shape and, in particular, its functionality as defined herein above, the filter device assembly implant of the present invention is capable of quantitatively or almost quantitatively capturing circulating tumor cells or metastatic cells or motile parts of tumor cells in a subject's body. Due to its shape and, in particular, its functionality as defined herein above the filter device assembly implant of the present invention is accordingly capable of preventing downstream organs or tissues to be reached by tumor cells or metastatic cells or motile parts of tumor cells which are circulating in a subject's body.

In further specific embodiments, the filter device assembly implant of the present invention is specifically designed to be implanted into a blood vessel. Examples of envisaged blood vessels are an artery, an elastic artery, a distributing artery, an arteriole, a capillary, a venule or a vein. It is further envisaged to design the filter device assembly implant for an implantation into a heart chamber or an elastic artery. The filter device assembly implant may accordingly be designed as free floating device with connecting wires to maintain its position. This concept also allows for retrieval. In further specific embodiments, the filter device assembly implant of the present invention is specifically designed to be implanted into a lymphatic vessel.

It is particularly preferred that the filter device assembly implant of the present invention is used by implantation in a blood or lymphatic vessel downstream of an existing cancer site in a subject. If such a positioning downstream of an existing cancer is envisaged, it is preferred that the filter device assembly implant is used by implantation in close proximity to said existing cancer site.

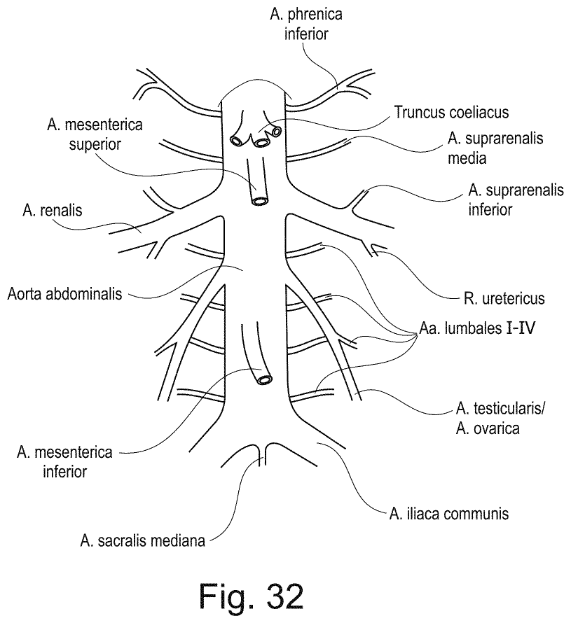

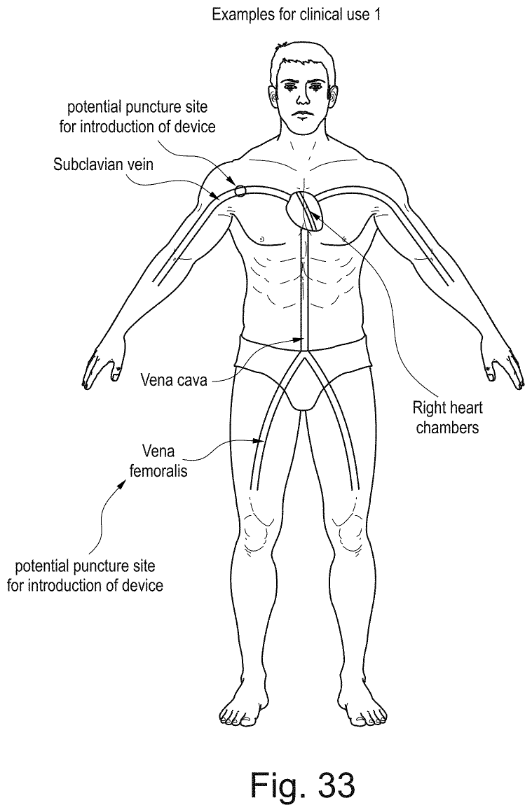

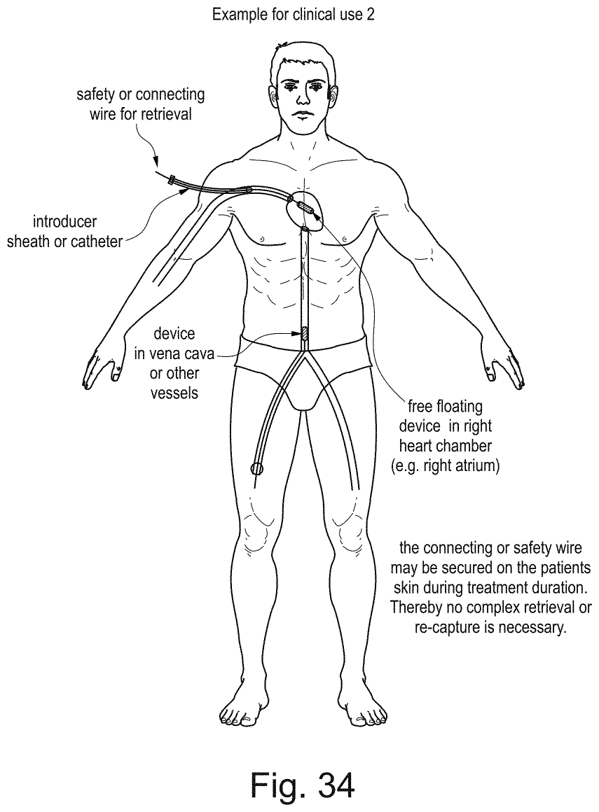

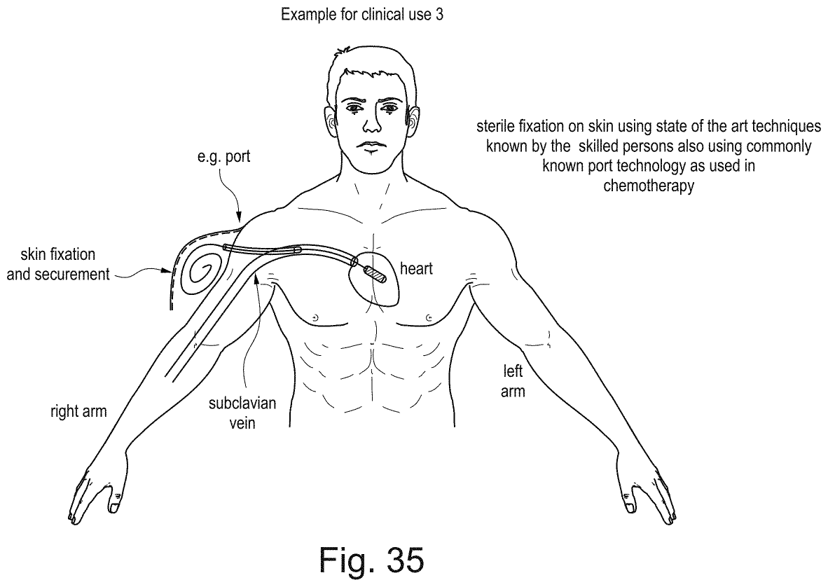

In further preferred embodiments that the filter device assembly implant of the present invention is used by implantation in a blood or lymphatic vessel upstream of a tissue with a high risk of developing metastasis. Further information may be derived, for example, from FIG. 32, which shows relevant blood vessels and mentions the connected tissues.

In yet another aspect the present invention relates to a method of treating cancer and/or metastasis, comprising implanting a filter device assembly implant according to the invention into a subject in need thereof. Also envisaged is a method of preventing cancer and/or metastasis, comprising implanting a filter device assembly implant according to the invention into a healthy subject or a subject being at risk of developing cancer and/or metastasis.

In a further aspect the present invention also envisages a method of manufacturing a filter device assembly implant as defined herein. The method preferably comprises the step of providing a biological agent by expressing said biological agent as polypeptide in a suitable host cell. Furthermore, the biological agent, e.g. a polypeptide, may be modified, e.g. by adding one or more of a sugar, a branched or unbranched multiple sugar structure, an alkyne, an azide, a streptavidin, a biotin, an amine, a carboxylic acid, an active ester, a epoxide or an aziridine. The method may further comprise coating steps which cover or at least partially cover the filter device assembly implant with a passive coating and/or an ECM-like structure and/or a biological agent or active coating.

The following examples and figures are provided for illustrative purposes. It is thus understood that the example and figures are not to be construed as limiting. The skilled person in the art will clearly be able to envisage further modifications of the principles laid out herein.

### EXAMPLES

### Example 1

Human pancreatic cancer cells were purchased from ATCC CFPAC-1 ATCC® CRL1918™ pancreas; derived from metastatic: liver; human origin. LOT:64094719. The human pancreatic cancer cell line was purchased from American Type Culture Collection (Manassas, Va.). Cells were thawed washed 2-3 times (1200 rpm, 3′, RT) and subsequently cultured in MDM (Life Technologies) supplemented with 10% FBS (Life Technologies) and PenStrep at 37° C. and 5% CO2 in a humidified incubator.

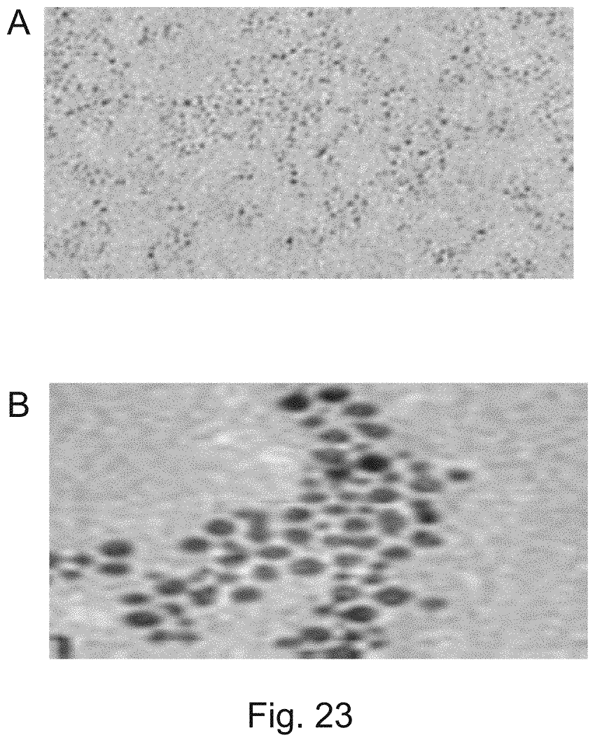

All of the experiments were performed with late passage cells. Cells grew in 3D-spheroids after 6-8 passages. After cell spit 1:10 cells were incubated in fresh medium and transferred to 12 well plate (see below). At day 5 spheroids were counted using 25× amplification under a microscope to establish an average number of spheroids. At day 5, CFPAC-1 cells and spheroids were incubated with Taxus Liberté, Paclitaxel-eluting coronary stent system Monorail (size 2.5 mm×20 mm) for another 10 days. At day 15 spheroids were counted under a microscope (see FIG. 23).

The coated stent was placed inside a well of a 12 well plate as mentioned above. Under normal conditions the splitted cells, with a cell scraper, immediately started to build 3D structures. These structures looked like branches, which turned into spheroid structures upon further culturing. Under these conditions approximately n=30 3D structures were counted at day 15 under a microscope. In the well with the coated stent, spheroid structures were markedly reduced at day 15 (see FIG. 23). There was still a monolayer of cells in the bottom of the well but the number of spheroids were less compared to the well without the stent. Under the microscope about 10-15 3D structures were counted. Many of those structures demonstrated tree- or finger-like branches, some spheroids were present, n=12. It is assumed that Paclitaxel slowed down growth capacity of CFPC-1 cells to generate 3-D spheroid structures.

In addition, RNA was prepared (RNeasy Plus Mini Kit, Qiagen) in order to investigate the transcription profile of cells+/−Paclitaxel treatment. RNA was stored at −80° C. Transcription profile investigation is conducted at state of spheroid generation in vitro.

### Example 2

In a separate experiment (data not shown) CFPC-1 cells were labeled with Cell Tracker Orange Fluorescent Probe from Lonza. Cell labeling was performed according to manufacturers protocol, Cat. No. PA-3012. Tumor cells express CA19-9 antigen. Using an antibody CA19-9 monoclonal antibody (121SLE), Thermo Fisher, tumor cells were labeled. Preoperative elevated CA-19-9 levels in patients with stage I pancreatic carcinoma decrease to normal values following surgery. When used serially, CA19-9 can predict recurrence of disease prior to radiographic or clinical findings. Antibody working solution was prepared according to manufactures protocol. SEFAR MEDIF AB 03-125/39 70-100 M 102 cm, SEFaR MEDIF AB 03-170/54 102 cm and SEFAR PETEX 07-6/5 115 cm and SEFAR PETEX 07-5/1 115 cm were submerged in anti CA19-9 antibody working solution. The membranes were first autoclaved and then incubated in solution for 1 hour at RT. The membranes were cut in app. 1 cm² and put in a tube (plastic straw).

The artificial device was submerged into a 12 well media solution containing the tumor cells. Using a 1 ml Pipette cells were flushed through the device 10-12 times within 10 hours. The membranes without the antibody “coating” did not retain as many tumor cells labeled with fCell Tracker Orange, compared to controls. Controls were membranes without antibody coating. The intensity of fluorescence was counted in a fluorescence microscope, Zeiss.

All of the experiments were performed with late passage cells. Cells grew in 3D-spheroids after 6-8 passages.

### Example 3

### In Vitro Experiments: Chandler Loop

The below described in vitro system is a ready to use device system for a loop with a maximum diameter of 200 mm consisting of a rotation unit, a removable inox loop cradle for polymer tubing loops tube connectors or accurate closing lap joints and a temperature-controlled water basin (see also British Journal of Pharmacology (2008) 153, 124-131). The chandler loop system enables simulation of extracorporal blood circulation (ECC). The Chandler loop method provides a reliable technique for examining the effect of compounds or devices on rtPA-induced lysis in vitro. It is a reliable method to evaluate potential alterations in rtPA-induced lysis at clinically relevant concentrations of OncoStent scaffold coated bioactive molecules. This evaluation is performed for toxicological evaluation of our medical devices prior to in vivo studies).

Further in vitro tests applied for our filter device assembly implants are being performed according to EMA Doc. Ref. EMENCHMP/EWP/110540/2007. Non-clinical testing requirements for the drug eluting stents, non-clinical pharmacokinetic testing. The Medical Devices Directive and its corresponding Guidelines state that in the case of implantable devices, active implantable devices and devices of Class III, evidence of the clinical performance and safety of a medical device is provided by means of clinical data.

In vitro cell culture experiments are performed using CRL1918 human pancreatic cancer cells of a liver metastasis. In cell culture binding experiments are performed to select the most bioactive composition for a coating of the filter device assembly implant according to the invention, e.g. CXCR2, CXCR4, PDL1, TRAIL, RXR activation compounds and Mg²⁺. Co-cultures are performed using human immune cells and whole blood.

### Example 4

### In Vivo Experiments: T Cell Deficient Pig Model (Oncology)

A particular pathogen free animal model is used for in vivo experiments. The filter device assembly implant for pancreatic cancer is developed considering the aspect of clinical relevance (EMA Doc. Ref. EMEA/CHMP/EWP/110540/2007). The filter device assembly implants according to the invention show equivalence with regard to anticipated clinical studies:

Clinically: used for the same clinical condition or purpose; used at the same site in the body; used in similar population (including age, anatomy, physiology); have similar relevant critical performance according to expected clinical effect for specific intended use.

Technically: used under similar conditions of use; have similar specifications and properties; viscosity, surface characteristics; be of similar design; use similar deployment methods (if relevant); have similar principles of operation (same implant material, same bioactive coating material), same size.

Biologically: use of same materials in contact with the same human tissues or body fluids.

3-month old T cell deficient pigs are injected intravenously with 300 Mio CRL-1918 human cells. Time points for acceptable pathological evaluation depend upon the specifics of DES (i.e., polymer and medicinal substance characteristics, elution kinetics, etc). The pig receives three the filter device assembly implants according to the invention at the time of xenografting. The filter device assembly implants are placed into different vessels according to major pathways of tumor cell dissemination.

### Placement of Filter Device Assembly Implants:

Truncus coeliacus (common port for three arteries coming from the Aorta adbominalis; Arteria splenica, Arteria gastroduodenalis) and Arteria mesenterica superior
Vena cava inferior
Venae pancreaticae, Vena splenica
Arteria lienalis

Further details can be derived from FIG. 32.

CRL-1918 human pancreatic cancer cells are transduced (Lipofectamine, nonviral transfection system) in order to allow for convenient and rapid analysis of circulating tumor cells in vivo.

### Experimental Design

Day 1: human CRL1918-GFP tumor cell inoculation, n=3×10⁸ cells, iv

Day 2: filter device assembly implant bioactive scaffold implantation and filter device assembly implant control implantation (non-active)

Day 0, 4, 8, 16, 30, 60: blood sampling, for biological characterization (expression of CA19-9 tumor marker, exosome formation, quantitative analysis and clinical chemistry)

Day 4, 8, 16, 30 and 60: ultrasound examination of the filter device assembly implant and vessel lumen (no thrombus formation)

Day 60: end of study, sacrifice, organ collection (liver, spleen, lung, brain, lymph nodes, intestine)

Day 0, 20, 40, 60: glucose tolerance test for pancreas functionality testing (IGTT)

### Biological Characterization:

Tumor cell analysis after blood collection is performed via FACS and ELISA to evaluate tumor cell surface marker expression and metastatic potential. TUNEL tests are performed to evaluate cell apoptosis potential. ADCC for immunological potential screen. Formation of exosomes (samples of days 30 and 60).

### Histology:

Staining Ki67 cell proliferation in liver tissue, CA19-9 staining in sampled organ tissue to evaluate metastatic spread. GFP measuring using fluorescence microscopy.

For evaluation of disease free survival non-active and active filter device assembly implants are being placed in separate animals.

### Filter Device Assembly Implant Specifications Used:

The following specifications are used for the experiments:

Size exclusion filter, pore sizes up to 100 nm diameter; filter device assembly implants to scavenge circulating tumor exosomes; material e.g. nitinol; no bioactive coating.

Size exclusion filter, pore sizes up to 100 nm diameter; filter device assembly implants to scavenge circulating tumor exosomes; material e.g. nitinol; with bioactive coating.

Size exclusion filter, pore sizes range from 70-100 nm diameter; filter device assembly implants to scavenge circulating tumor exosomes; material e.g. nitinol; no bioactive coating.

Filter device assembly implants of different matrices regarding design and material (e.g. carbon) and e.g. onion-shaped membranes as scaffolds.

Preferably, filter device assembly implants as described in FIGS. 24 to 31 and explained in the corresponding passages of the description, above, are used.

Combination of up to three different filter device assembly implants with respect to size exclusion in addition to bioactive molecule selection and coating of the matrix

Combination of up to three different filter device assembly implants with respect to size exclusion in addition to bioactive molecule selection and coating of the matrix and drug elution e.g. Mg²⁺.

Filter device assembly implants with bioactive immune-molecule coating to modify circulating tumor cells (Il-10, PDL1 etc.).

Filter device assembly implants with bioactive proapoptotic molecule coating (FAS, FASL etc.).

Filter device assembly implants with cell coating to allow for specific viable interaction with circulating tumor cells.

Filter device assembly implants with bioactive smart peptide coating to tag circulating tumor cells.

Filter device assembly implants with cytokine reservoir to attract circulating tumor cells.

Filter device assembly implants with combination of bioactive coating of cells, cytokine gradient and peptide tags.

1. A filter device assembly implant comprising one or more chemical and/or biological agents wherein the implant is capable of recruiting a circulating tumor or circulating metastatic cell or motile parts of tumor cells and thereby removes said cell or motile part thereof from circulation, wherein said implant has one or more of the following properties: (i) it is catheter based; (ii) it is freely positionable in a target vessel, preferably in a minimal invasive manner; (iii) it is retrievable, preferably by catheter means and/or in a minimal invasive manner; (iv) it is anchorable in a target vessel; (v) is designed to fit into and be connected to a permanent implant present in a target vessel as a shuttle docking to a receiving site.

2. (canceled)

3. The filter device assembly implant of claim 1, comprising (i) at least one a reversibly expandable device body having a proximal and a distal end and (ii) at least one a filter membrane, preferably characterized by the presence of pores, wherein said pores have preferably a pore diameter which ranges from about 7 μm to about 100 μm, more preferably in a differential manner.

4.-6. (canceled)

7. The filter device assembly implant of claim 1, wherein said implant is provided in a tubular, onion like, pearl-chain-like, or a birds-nest like shape, or in any mixture of these shapes, wherein preferably said shape is provided by an elastic memory shape meshwork.

8.-11. (canceled)

12. The filter device assembly implant of claim 2, wherein said implant is self-expandable, wherein preferably at least a portion of the implant can be activated by balloon inflation.

13.-14. (canceled)

15. The filter device assembly implant of claim 2, wherein said implant comprises a radiopaque marker, wherein said radiopaque marker is preferably located at the proximal and distal end, or on at least two opposite portions of the outermost structural element of the device body, allowing to judge radial expansion under medical imaging.

16.-20. (canceled)

21. The filter device assembly implant of claim 2, wherein said implant comprises at least two membranes, each of which incompletely covers the cross-sectional area, and which are arranged in tandem position along the longitudinal axis of the device body, preferably opposite to each other within the circumference of the device body or shifted in clockwise orientation in case of more than 2 membranes in tandem position, or comprises alternating non-completely covering filter membranes, preferably in a pearl-chain, onion type or birds-nest like shape.

22. (canceled)

23. The filter device assembly implant of claim 2, wherein said filter membranes have differential pore diameters and/or differential pattern, preferably ranging from about 7 μm to about 100 μm, or wherein two or more filter membranes have differential pore diameters and/or differential pattern, preferably ranging from about 7 μm to about 100 μm.

24. The filter device assembly implant of claim 1, additionally comprising a retrievable embolic filter, preferably with a pore diameter of >100 μm.

25. The filter device assembly implant of claim 2, wherein said at least one filter membrane is fully or partially coated on its interior side; or on its exterior side; or on both sides with said one or more chemical and/or biological agents; or wherein said coating differs between different filter membranes, wherein said coating is a passive coating with one or more polymeric materials such as ethylene vinyl acetate (EVA), latexes, urethanes, polyurethanes, polysiloxanes, styrene-ethylene/butylene styrene block copolymers (SEBS), polytetrafluoroethylene (PTFE) or linear aliphatic polyesters.

26.-29. (canceled)

30. The filter device assembly implant of claim 1, wherein said one or more chemical and/or biological agents constitute an extracellular matrix-like structure, wherein said chemical and/or biological agents constituting an extracellular matrix-like structure are preferably selected from the group comprising proteoglycans, such as heparan sulfate, chondroitin sulfate and/or keratin sulfate; non-proteoglycan-polysaccharides such as hyaluronic acid; collagen; elastin; fibronectin and laminin, or a mixture thereof; preferably a protein mixture secreted by Engelbreth-Holm-Swarm (EHS) mouse sarcoma cells, Matrigel, BioCoat or GelTrex.

31. The filter device assembly implant of claim 1, wherein said implant provides an environment for circulating metastatic cells.

32. The filter device assembly implant of claim 1, wherein said implant comprises a biological agent as an active coating, which is capable of binding to a tumor marker, wherein preferably said tumor marker is CCR4, CCR6, CCR7, IGF, LFA-1, VLA-4, VLA-5, CD44, CD44 v4-v7, CD44 v6-v7, CD44 D3 (v6-v7), CD44-R (v8-v10), CD44 v10, CD-44R1, CXCR3, CXCR4, CXCR5, CXCR6, CXCR7, Surface Fibronectin, PECAM-1 (CD31), CAM 120/180, Integrin alphav beta5, P-Selectin, L-Selectin, Integrin alphav beta5, Integrin alpha4 beta7, Integrin alpha2 beta1, Integrin alpha2 beta3, Integrin alphav beta3, Galectin-3, N-CAM, L-Selectin, LPAM-I (alpha4 beta2), CTLA, Integrin alpha4 beta1, Integrin alphaE beta7, CCR10, Axl/Mer, Anxa2-R or Desmoglein I (DG I).

33.-34. (canceled)

35. The filter device assembly implant of claim 12, wherein said biological agent which is capable of binding to a tumor marker is selected from one or more of the group comprising a tumor-marker specific antibody or a fragment thereof, CD133 or a fragment or domain thereof, VEGFR-1 or a fragment or domain thereof, a homing factor or a fragment or domain thereof and a tumor-marker specific lectin or a fragment or domain thereof.

36. (canceled)

37. The filter device assembly implant of claim 12, wherein said biological agent is linked to the passive coating of the implant or to the structural support material via a spacer element, wherein said spacer element is preferably composed or partially composed of a peptide or polypeptide, preferably the Fc part of an antibody or multi-histidine tag; a nucleic acid; a modified nucleic acid; or a polymer such as PEG, PLA, PVA, polyethylene or polypropylene.

38.-41. (canceled)

42. The filter device assembly implant of claim 12, wherein said biological agent comprises, essentially consists of, or consists of a binding domain capable of binding to a tumor marker, wherein said binding domain is peptide or polypeptide molecule having a length of about 20 to about 250 amino acids, preferably of about 20 to about 120 amino acids.

43.-44. (canceled)

45. The filter device assembly implant of claim 15, wherein said biological agent additionally comprises one or more functional domains, preferably an apoptosis inducing factor or a functional domain of an apoptosis inducing factor capable of inducing apoptosis.

46.-51. (canceled)

52. The filter device assembly implant of claim 12, wherein said biological agent is provided as linear or circular element or as an element composed of linear and circular parts, preferably as a linear or circular or partially linear/circular peptide or polypeptide, wherein said circular biological agent preferably has or is part of a structure comprising a loop or a loop and a stem; or of a linear structure, which is linked to said spacer element.

53.-72. (canceled)

73. A method of treating cancer and/or metastasis, comprising implanting a filter device assembly implant as defined in claim 1 into a subject in need thereof.

74. A method of preventing cancer and/or metastasis, comprising implanting a filter device assembly implant as defined in claim 1 into a healthy subject or a subject being at risk of developing cancer and/or metastasis.

75. A method of manufacturing filter device assembly implant as defined in claim 1, preferably comprising the step of providing a biological agent as defined in claim 12 by expressing said biological agent as polypeptide in a suitable host cell.

76. (canceled)
